(19) United States
(12) Patent Application Publication
(10) Pub. No.: US 2003/0032003 A1
(43) Pub. Date: Feb. 13, 2003

(54) IN VIVO SELECTION

(76) Inventors: ROBERT H. SCHIESTL, BOSTON, MA (US); JIRI AUBRECHT, MYSTIC, CT (US)

Correspondence Address: KENNETH I KOHN, KOHN & ASSOCIATES, 30500 NORTHWESTERN HIGHWAY, SUITE 410, FARMINGTON HILLS, MI, 48334

(21) Appl. No.: 09/402,130

(22) PCT Filed: May 22, 1997
(86) PCT No.: PCT/US97/08788

(51) Int. Cl. C12Q 1/70; A01K 67/00; C07H 21/04; C12N 15/87

(52) U.S. Cl. 435/5; 435/455; 435/325; 800/8; 800/21; 536/23.1

(57) ABSTRACT

A method of selecting for cells in vivo is disclosed. The method includes the steps of providing in vivo cells which are resistant to a selecting substance and cells non-resistant to the selecting substance. Resistant cells are selected for in vivo by providing the selecting substance in vivo at a dose nontoxic to the resistant cells and toxic to the nonresistant cells thereby replacing nonresistant cells by resistant replacement cells

### BACKGROUND OF THE INVENTION

1. Technical Field

This invention relates to methods of selecting in vivo for cells and organisms containing predetermined genes.

2. Background Art

In transgenic technology presently, to obtain targeted integration events the method of choice for generation of transgenic animals comprises the use of embryonic stem cells (ESC). These ES cells are transfected in culture with selectable marker genes and clones bearing the desired manipulation are selected for. Investigators frequently use positive selection (e.g. for neomycin or hygromycin resistance, see for example U.S. Pat. No. 5,219,740) for integration events including targeted as well as random integrations. At the same time negative selection (e.g. with gancyclovir against the Htk gene, see U.S. Pat. No. 5,482,837 for other examples) is used to select against random integration events. This scheme is called positive-negative selection or PNS. This method is limited by the ability of the embryonic stem cells (ESC) to contribute to the embryonic tissues and to produce chimeras with germ-line transmission of the desired manipulation. U.S. Pat. Nos. 5,487,992 and 5,464,764 describe such processes and are incorporated herein in their entirety by reference. As demonstrated by this method, the production of chimeric animals and screening for germline transmission currently takes a significant amount of time and large numbers of animals.

To obtain random integrations, current methods utilize the injection of recombinant DNA into fertilized eggs. All cells of offsprings will contain the transgene that developed from one egg that has incorporated such a transgene. This avoids the tedious phase of producing chimeras and germline transmission. However, this approach is not used for targeted integration because of the low frequency of homologous integration (1/20 to 1/40,000). U.S. Pat. No. 5,487,992 provides a listing of transgenic animals produced by random integration as set forth in column 1 of the patent as well as problems associated with random integration. The major limitation of this procedure is again the necessity to examine large numbers of offspring.

An improvement of both of the above integration techniques is the use of yeast artificial chromosomes (YAC) which allow the transfer of large cloned chromosomal regions containing the genomic DNA with introns and essential regulatory sequences of a target gene instead of cDNA with non-specific regulators (for a review see Lamb and Gearhart, 1995). The use of YACs for random integration particularly overcomes some of the problems associated with other vectors for random integration set forth in the '992 patent.

It is an objective of the present invention to provide a method of selecting for successful random integration events. It is a further objective of the present invention to provide a method for selecting for successful targeted integration events in vivo and against random integration events thereby eliminating the necessity to examine large numbers of offspring. Further, it is an objective of the present invention when using chimeric animals to increase the selection efficiency for germline transmission in such animals.

Transplantation

The field of organ transplantation has had increasing success in the past 25 years due primarily to improved strategies to overcome allorejection. However, this has raised a new problem—the shortage of donor organs. Xenotransplantation (i.e. across species lines) would solve the problem of a supply of donor organs, particularly if the donor is an animal currently raised for food.

Graft rejection is the most serious obstacle for the application of xenotransplantation in clinical practice (for review see Kaufman, et al., 1995). Rejection has two phases. The fast acute phase of xenorejection, also called hyperacute rejection (HAR), is caused by binding of natural antibodies (mostly IgM) to the graft endothelial cells and activation of the complement pathway (Gambiez et al., 1992; Platt et al., 1990). This can be avoided by either removing natural antibodies (Tanaka et al., 1994; Johnson et al., 1992), by interfering with complement activation (Weisman et al., 1990; Fodor et al., 1994) or by genetic engineering of the donor organism (for review see Cozzi and White, 1995) so that complement binding sites are removed.

The later phase of xenorejection is mediated by cells, most probably CD4⁺ T cells (Pierson et al., 1989). Recent progress in development of potent immunosuppresives particularly Cyclosporine and FK506 has brought progress in regulation of the cell mediated response. However, long term immunosuppressive therapy causes serious side effects (Goodman and Gilman's).

Induction of tolerance to the xenograft can be achieved by xenogeneic chimerism (for review see Kaufman, et al., 1995). This approach involves transient ablation of the recipient's immune system, during which time partial donor hematopoietic repopulation is established by bone marrow transplantation. The immune system then reconstitutes in the presence of both recipient and donor hematopoietic cells, and becomes tolerant to both (Sharabi et al., 1990; Ildstad et al., 1991).

U.S. Pat. No. 5,192,312 provides a method of treating xenogeneic transplantable tissue ex vivo or in vitro prior to transplant to extend graft survival. However, this method requires repopulating the xenograft with allogeneic cells through tissue culture and is therefore of limited value in transplanting organs as well as preparing large amounts of tissues for transplant.

Bone marrow xenotransplantation

Bone marrow xenotransplantation has great potential in clinical practice. As mentioned above the induction of tolerance can be achieved by xenogeneic chimerism (for review see Kaufman, et al., 1995). On the other hand, production of blood derivatives in non-human animals such as platelets, white and red blood cells requires complete repopulation of the animals hematopoietic system with human stem cells. Human hematopoietic cells can survive in sheep for long periods of time (more than 2 years) (Zanjani et al., 1991), however the resulting bone marrow was found to be chimeric. Interestingly, human hematopoietic stem cells from these chimeric sheep were able to grow in new sheep recipients (Zanjani et al., 1994). This suggests that human hematopoietic cells can survive and be passed from one xenogeneic recipient to another. It would be useful to have fully transgenic large animals, rather than chimeric animals now available, as a source of human bone marrow for transplant as well as being “factories” for human blood derivatives and as organ donors. Further these animals could also be used as a model system for drug testing.

Pig organs have been proposed as the system of choice (Cozzi and White, 1995) for providing xenotransplants because of porcine anatomical and physiological similarities to humans, as for example in the fat to muscle ratios and blood groupings. It is an objective of the present application to provide a method of selecting for fully transgenic animals in vivo to be used as organ donors.

HIV Infection

In general, HIV infection runs in cycles. Usually, the immune system attacks and eliminates most infected lymphocytes. However, some infected cells contain subpopulation of viruses with changed antigenicity and thus evade the immune system. They are the source for new viral infection of newly developed lymphocytes. In the next cycle of the HIV infection the immune system recognize this subpopulation and destroys most of infected cells. However, new subpopulation of viruses appear and the cycle repeats. At the end the immune system is outperformed by the speed of viral variability and looses its capability to reduce the infection and the disease approaches the terminal stage.

One proposed therapy is to replace the human bone marrow with a xenotransplant that is not susceptible to HIV. Another proposed therapy is to destroy cells carrying the virus. However, xenotransplants involve severe risks of rejection (Kaufman et al., 1995) so that novel methods of allowing xenotransplantation are needed. As discussed above, the rejection reaction in xenotransplants is vigorous and not adequately controlled by currently available immunosuppressive drugs. Currently, there are several HIV infected patients who received transplanted baboon bone marrow. The baboon is resistant to HIV. However, baboon hematopoiesis in these patients has not been established (Ricordi et al., 1996).

It is an objective of the present invention to provide selectable xenotransplants that are not rejected, generally are resistant to HIV infection. It is another objective of the present invention to provide bone marrow cells that can be killed should they become infected.

Chemotherapy

Cytotoxic drug resistance is a major obstacle to successful chemotherapy in cancer patients. 6-Thioguanine (6TG) is effective as leukemia treatment agent as well as immunosupressant (Loo and Nelson, 1982; Calabresi and Parks, 1985). It has been noted that virtually all major current protocols for “average” and “low risk” acute lymphoblastic leukemia (ALL) include as a core component of continuing chemotherapy (Lennard and Lilleyman, 1985) daily doses of 6-mercaptopurine (6MP), an analog of 6TG that is metabolized in the same way (Calabresi and Parks, 1985; LePage, 1963; Pan and Nelson, 1990; Ling, et al., 1992; LePage and Whitecar, 1971; Elion, 1967).

Several laboratory and clinical observations suggest that Hprt deficiency causes cellular resistance to 6TG. For example, cells from Lesch-Nyhan syndrome patients lack Hprt and are also resistant to 6TG (Dempsey, et al., 1983; Yamanaka, et al., 1985.). Most chemically induced mutant cells that are resistant to 6TG show significantly reduced Hprt activity (Sato, et al., 1972; reviews: Siminovitch, 1976; Caskey and Kruh, 1979). Many leukemia patients treated with 6MP develop 6MP resistance; hence the drug fails to maintain remission of the disease (Brockman, 1974). Among 15 analyzed cases of 6MP resistant leukemias one was due to complete loss of Hprt activity (Davidson and Winter, 1964). Additional mechanisms of such 6TG resistance include lower affinity of Hprt for the ribose-phosphate donor 5′-phosphoribosyl-1-pyrophosphate (PRPP), increased degradation of 6MP, decreased incorporation of the analog into polynucleotides, and failure of the analog to enter the cells (Brockman, 1974). Prevention of 6TG toxic effects can be achieved by administration of the purines adenine or hypoxanthine in leukemia cells (Hashimoto, et al., 1990) or by adenosine in vitro and in vivo (Epstein and Preisler, 1984). This protective effect has been explained as a decrease of 6TG bioactivation by competition for PRPP (Hashimoto, et al., 1990), or by its depletion (Epstein and Preisler, 1984).

Although the correlation between Hprt deficiency and 6TG resistance is established very well in cultured cells (Sato, et al., 1972; reviews: Siminovitch, 1976; Caskey and Kruh, 1979) there exist little or no data which address in vivo levels of Hprt activity and whole animal or tissue specific 6TG toxicity. Hprt might be involved in toxic side effects of 6TG therapy and relative or absolute Hprt deficiency could play an important role in the development of 6TG resistant tumors.

It is an objective of the present invention to determine a method of identifing patients who are being treated with 6TG analogs which are at risk of developing cytotoxic drug resistance.

### SUMMARY OF THE INVENTION AND ADVANTAGES

According to the present invention, a method of selecting for cells in vivo is disclosed. The method includes the steps of providing in vivo cells resistant to a selecting substance and cells non-resistant to the selecting substance. Further the method then selects in vivo for the resistant cells by providing the selecting substance in vivo at a dose nontoxic to the resistant cells and toxic to the non-resistant cells thereby replacing non-resistant cells by resistant replacement cells.

In an embodiment of the method of the present invention, cells are genetically engineered to be resistant to a selecting substance, the cells in vivo are implanted and the selecting substance is administered at a dose nontoxic to the engineered cells but toxic to non-engineered cells, thereby allowing the engineered cells to replace the non-engineered cells.

In a further embodiment, the method is directed to a method of selecting for cells in vivo by identifying a cell population in a host that carries a gene making the host cell population selectively susceptible to a compound. Replacement cells that are not susceptible to the compound are administered to the host to replace the targeted cells. The compound is either co-administered with the cells or subsequently administered at a dosage that is selectively toxic for the host cell type but not the replacement cells.

The method of the present invention is applicable in xenotransplantation, cancer therapy, autoimmune therapy, genetic disorders as for example SCID, AIDS therapy and development of transgenic animals for therapeutic drug testing and as a source of human compatible cells, tissue and organs.

### DETAILED DESCRIPTION OF THE PREFERRED EMBODIMENT

According to the present invention, a method of selecting for cells in vivo is disclosed. The method includes the steps of providing in vivo cells resistant to a selecting substance and cells non-resistant to the selecting substance. Further the method then selects in vivo for the resistant cells by providing the selecting substance in vivo at a dose nontoxic to the resistant cells and toxic to the non-resistant cells thereby replacing non-resistant cells by resistant replacement cells. The selecting step may be repeater.

The step of “providing, in vivo, cells which are resistant to a selecting substance,, can be accomplished most generally by either in vivo manipulation of cells present in an organism or administration of cells that either inherently possess a resistance to a selecting substance or have been altered either by genetic manipulation known in the art or by drugs or other means to impart a resistant trait to a selecting substance. Examples of such are given below.

Provision of the resistant cells to a host, such as an animal, a patient, or the like, creates an environment wherein a host organism possesses cells which are both susceptible or non-resistant to a selecting substance and also cells resistant to the selecting substance. The selecting substance is then administered to the host, or induced within the host, at a dose non-toxic to the resistant cells but toxic to the targeted or selected population of non-resistant cells resulting in the selection for the resistant cells. In preferred embodiments, this can result in replacement of the targeted non-resistant cells by the resistant cells.

The term, “non-resistant” is used to mean cells which are susceptible to cell death or at least becoming afunctional or nonfunctional in the presence of the selecting substance at a dose which is not toxic to the functioning of the resistant cells. Likewise, the resistant cells, as a population, will survive administration of, and the presence of, a selecting substance. That is, the resistant cells will functionally survive the applied dose of the selecting substance.

By “selecting in vivo”, it is meant that the operative selection step occurs within a recipient. The terms “tissue”, “organ” and “organ part” are used in a general sense herein to mean any transplantable or implantable tissue, organ or organ part or cellular matrix from the tissue, organ or organ part. The term “cells” is used also as a general term to encompass cells, tissue, organs as will be apparent from the context since both tissues and organs contain cells and the cells are the ultimate target of the present invention. The term “transplant” and “implant” are used herein (and interchangeably) to refer to any cells, tissues, organs or organ parts which can be transplanted, i.e. introduced into a recipient to replace or supplement the structure or function of endogenous tissue. The term “genotype” can refer to one or more genes that control a trait being monitored. The term “autologous” refers to tissue or cells which originate from the recipient. The term “allogeneic” and/or “allograft” refer to cells, organs and/or tissues which originate from donors of the same species as the recipient. The term “xenograft” refers to cells, organs and/or tissues which are derived from a species other than that of the recipient.

The selecting substance can be a compound, drug, naturally occurring or synthetic product, or the like which when administered in vivo renders the non-resistant cells at the least afunctional or non-functional. The selecting substance is toxic to a targeted non-resistant cells but is non-toxic to a group of resistant cells as discussed herein. As discussed herein, the selecting substance can be a toxic drug, a naturally occurring product which is administered to the host or it can be a naturally occurring product which is produced in vivo (or not produced) by the host to act upon the two populations of cells (resistant and non-resistant cells) as discussed above. Hence, it could be a naturally occurring hormone, blood factor, antibody or the like and which in an embodiment can be induced or suppressed. Such materials are known in the art. In a further embodiment, the selecting substance can be in the form of a bacterial, viral, or other pathogen which will select for replacement of non-resistant or susceptible cells thereby resulting in replacement of such cells by resistant cells. The resistant cells are selected to functionally replace the non-resistant cells thereby resulting in a functional cure of a pathogenically induced disease state.

The terms “positive selection” and “negative selection” are used herein as follows. In positive selection a marker that is (a) either not expressed (not present) in the host, or (b) which is not sufficiently expressed to give resistance to the selecting drug is employed. Expression in case of (a) or overexpression in case of (b) of these markers confers resistance of the overexpressing cells or with at least reduced susceptibility to the selecting compound. Markers in the (a) category are usually markers from another organism, in general resistance markers are used, such as neo, hyg, pac, ble, zeo, mdr1, hisD expressed under mammalian promoters. Markers in the (b) catagory such as DHFR, CAD or MDR1 are endogenous genes that are overexpressed under a strong promoter resulting in resistance of the modified cells.

In Negative selection two alternatives are possible. In (a) a marker such as Hprt that is commonly present in the host cells confers sensitivity to a particular selective drug by metabolizing the drug to a toxic substance. Inactivation (mutation, deletion) of such marker leads to drug resistance. In (b) a marker from another organism such as HSV-TK from herpes simplex confers sensitivity to a certain drug. Host cells are resistant, cells expressing HSV-TK are sensitive. Table 12 sets forth examples of such markers in positive and negative selection.

The selecting substance, compound or drug in one embodiment is 6-thioguanine (6TG) or alternatively 8-azaguanine, 6-mercaptopurine or other purine analogs which become selectable in hosts that have an appropriate mutation in the Hprt gene.

Further selecting compounds are listed in Table 12 as well as diphtheria toxin, ouabain, and anti-HLA antibody. These selecting substances and others can be used to select cells in which a mutation renders them resistant to the compound while the non-mutant cells are susceptible. In general, 6-Thioguanine dosage and administration in humans is 2 mg/kg of body weight per day p.o., it can be increased to 3 mg/kg of body weight per day. (Tabloid brand Thioguanine, PDR, Medical Economics Data Montenvale, N.J. 1993 (47th edition) and forward pp.835-7) Dosages and patient treatment protocols are generally as set forth in the Physicians' Desk Reference (PDR) current edition.

In the present invention, the selecting substance/compound in general will be toxic to the susceptible cells at a dose lower than that of the resistant cells. Further the compound will be capable of being administered in vivo at a dose that is toxic to the susceptible cells but that is not toxic to the recipient as a whole as shown in the Examples herein below.

The following embodiments of the present invention exemplify its wide availability for use in various areas of cell selection, disease control, and the like. As an exemplar of the present invention the Hprt/6TG system will often be used in the following discussion but it is to be understood that any selecting compound which will provide the same results will be capable of being used in the present invention.

The method can, in general, include genetically engineering the cells to be resistant to a selecting substance or compound, implanting the cells in vivo and administering the compound at a dose nontoxic to the engineered cells but toxic to non-engineered cells, thereby allowing the engineered cells to replace the non-engineered cells. Additionally, it is possible to genetically engineer a cell population in the host to be susceptible to a selecting substance so that the method can be practiced. Any known methods of genetic engineering or gene therapy can be used to prepare the cells.

In a further embodiment, the method is directed to a method of selecting for cells in vivo by identifying a cell population in a host that carries a gene making the host cell population selectively susceptible to a substance, compound or drug. Replacement cells that are not susceptible to the compound are administered to the host to replace the targeted cells. The compound is either co-administered with the cells or prior to or subsequently administered at a dosage that is selectively toxic for the host cell type but not the replacement cells. In one embodiment the replacement cells are xenotransplants. In a further embodiment the replacement cells have been genetically engineered to also carry at least one transgene.

The present invention provides a method of selecting for transgenic animals in utero by selecting against fertilized eggs in utero that are genetically susceptible to a selecting substance. In the method donor fertilized eggs are injected with a vector carrying a transgene and means for inactivating the drug sensitivity. That is the donor eggs are from a genetic background that is sensitive to the selecting substance and the transgene is linked on the vector to a gene that confers resistance to the selecting substance.

In one embodiment, the gene that confers resistance could be under the control of a regulatory promoter. Alternatively, antisense technology can be used to confer the resistance by having the antisense sequence for the selecting substance sensitivity gene in the vector. The injected fertilized eggs are implanted in a pseudopregnant female who in one embodiment is itself carrying the selecting substance resistant gene. However, depending on the selecting substance used, and required dosage, it is possible to use females for implantation that are also sensitive (non-resistant) to the selecting substance. The implanted female is then treated with the selecting substance thereby selecting for embryos that are resistant and which carry the transgene and selecting against embryos that are sensitive and which do not carry the transgene.

Using negative selection, the selecting substance can be 6-thiguanine (6TG) or alternatively 8-azaguanine, 6-mercaptopurine or other purine analogs which become selectable in hosts that have an appropriate mutation in the Hprt gene. Further selectable compounds are diphtheria toxin, ouabain, and anti-HLA antibody. These selecting drugs and others (see Table 12) can be used to select cells in which a mutation renders them resistant to the compound while the non-mutant cells are susceptible. Alternatively, hygromycin-B and G418 (neomycin analog) utilizing positive selection can be used. These selecting drugs and others can be used to select cells in which a selecting marker is added.

The present invention provides a method to establish chimeric animals which carry a transgene conferring resistance to a selecting substance with germ-line transmission in utero. In this method embryonic stem cells (ESC) are prepared carrying a transgene conferring resistance to the selecting substance. The ESC are implanted producing chimeric animals. To test for germ-line transmission the chimeric animals are mated and the chimeric female carrying fertilized eggs with possible germ-line transmission of the transgene are treated with the selecting substance. In one embodiment, depending on the selecting substance used, and required dosage, it is possible to use females for implantation that are also sensitive (non-resistant) to the selecting substance. Alternatively, the selecting substance can be delivered so as to be targeted to the embyro only. This selects for embryos that carry the gene conferring resistance to the selecting substance and selects against embryos that do not carry the transgene.

This method can be further used for the selection of germ-line transmission events in chimeras. As described above the chimeric animals are established and mated. The animals with possible germ-line transmission of the transgene are treated with the selecting substance. As discussed above, the dosing and delivery of the selecting substance must take into account that the chimeric animal will also be sensitive to the selecting substance.

Using negative selection, the selecting substance can be 6-thiguanine (6TG) or alternatively 8-azaguanine, 6-mercaptopurine or other purine analogs which become selectable in hosts that have an appropriate mutation in the Hprt gene. Further selectable compounds are diphtheria toxin, ouabain, and anti-HLA antibody. These selecting drugs and others (see Table 12) can be used to select cells in which a mutation renders them resistant to the compound while the non-mutant cells are susceptible. Alternatively, hygromycin-B and G418 (neomycin analog) utilizing positive selection can be used. These selecting drugs and others can be used to select cells in which a selecting marker is added.

These methods allow the establishment of transgenic animal models for testing. For example, as shown in the Examples herein, Hprt− transgenic animals can be established. Additionally, applicants have cloned the Hprt minigene (lacking introns) behind the myelin basic protein (MBP) promoter that is exclusively expressed in Schwann cells and behind the CD4 pTexSil promoter that is exclusively expressed in immature and mature CD4+ T cells. These constructs are then established in Hprt− transgenic mice by the methods of the present invention. Therefore in these animals, the hprt+ gene will be exclusively expressed in Schwann cells for the construct with the MBP promoter or in CD4 cells for the construct with the CD4 pTexSil promoter. Treatment with 6TG leads to ablation of Schwann cells or CD4 cells respectively. Therefore these models can be used for studying the severity, timing of onset, “healing”, or chronic expression of peripheral myelin dysfunction and lack of CD4+ cells in the animal model. Each aspect can be directed by the timing and/or dose or chronic treatment with different doses of 6TG. This approach can be used to destroy different kinds of immune cells and to determine the effect of such destruction on different aspects of the immune response.

The regeneration of the affected tissue may also be studied after discontinuation of the 6TG treatment. For instance, after whole body irradiation of an Hprt mutant animal the immune system may be reconstituted with cells from an Hprt wildtype (Hprt+) animal. Thereafter, different doses of 6TG could be used to specifically affect the immune system and to study regeneration. Developmental processes can be studied by expression of the Hprt minigene under a tissue specific promoter and treatment of the pregnant dam with 6TG would be undertaken at different timepoints in embryo development.

In general the murine system is used as the animal system but other experimental animals can be used to establish animal models as dictated by the disease and/or cells being studied as is known to those skilled in the art.

The present invention also provides for a method of establishing genetically engineered xenotransplant donors allowing graft acceptance by a human host of transplants across species lines. The method includes the establishment of transgenic animals carrying genes providing for a genotype (i.e. HLA haplotypes) that would express cell surface markers that would not be rejected by the human immune system. The genes could include those for the HLA as well as other species recognition genes. Pregnant females are established carrying embryos using transgenic technology (either random or targeted insertion) with the possibility of carrying at least one fertilized egg having a genotype allowing graft acceptance by a human host linked to a gene conferring resistance to a drug. The female carrying the fertilized eggs is then treated with the drug thereby selecting for embryos that carry the gene conferring resistance to a drug and which therefore carry the genotype allowing graft acceptance by a human host and selecting against embryos that do not carry the transgene. These animals are then a source of organ/tissue donors for human-compatible xenotransplants. In one embodiment pigs are used as the donor source.

Bone marrow transplantation is a common procedure in the treatment of many diseases. The success depends on the ability of donor stem cells to overgrow irradiated host's cells. As further provided by the present invention a method of selecting for bone marrow transplant cells in vivo is disclosed.

The method includes identifying a gene in a bone marrow recipient that makes the recipient's bone marrow population selectively susceptible to a compound. Replacement bone marrow that is not susceptible to the compound is then administered. The host/recipient is then administered the compound at a dosage that is selectively toxic for the recipient's bone marrow but not the replacement bone marrow.

In a preferred embodiment the replacement bone marrow is marrow stem cells from a Lesch-Nyhan patient since Lesch-Nyhan patients are Hprt−. This provides an added advantage in that 6-thioguanine is an immunosuppressive agent so that the rejection reaction will be diminished. In a further embodiment the marrow stem cells from a Lesch-Nyhan patient have been transformed with a MDR1 gene conferring multiple drug resistance to chemotherapy drugs.

Applicants have shown that Hprt wildtype hematopoietic cells are uniquely sensitive to 6TG whereas Hprt mutant hematopoietic cells are resistant. Moreover, the Hprt deficient bone marrow cells show hyperproliferation making it even more likely that they replace the necrotic Hprt+ cells.

Treatment of leukemia is currently carried out by whole body irradiation or chemotherapy. One limiting factor is immunosuppression as well as induced drug-resistance to the chemotherapeutic drugs as discussed hereinabove. The present invention provides for the transplant of bone marrow stem cells from a Lesch-Nyhan patient (Hprt deficiency) and to gradually replace the entire immune system of the cancer patient after several doses of 6TG. As applicants have shown, this is possible since Hprt deficient cells actually hyperproliferate in response to 6TG treatment (see Examples herein below). Once the bone marrow consists mostly of Hprt deficient cells immunosuppression by 6TG is not any more the limiting factor and higher doses of 6TG may be used. In addition, the bone marrow stem cells may be replaced with bone marrow stem cells from a Lesch-Nyhan patient that have been transformed with the MDR1 gene conferring multiple drug resistance. Thus, the bone marrow cells gain resistance to a wide variety of antitumor agents allowing more aggressive chemotherapy.

This also would be useful in gene therapy in that the transformed cells could carry a gene for other beneficial modification. For example, the Ada+ gene in Ada deficient recipients. Successful gene therapy trials are currently underway with Ada+ modified cells. However, the replacement takes several years during which conventional therapy might fail. This replacement could be accelerated if Ada+ Hprt deficient cells (from a Lesch-Nyhan syndrome patient) would be used coupled with 6TG treatment. The method can allow for a more rapid, yet gradual, cellular replacement. Additionally, children with other genetic disorders that require cellular replacement, particularly immune deficiency disorders could be treated with the present invention.

Generally, treatment for autoimmune diseases such as Myasthenia gravis includes long-term immunosuppression with steroids and other agents. Such treatment protocols have long-term side affects due to the steroids and also expose the patient to the possibility of severe infections due to the generalized suppression of the immune system. The bone marrow transplant of the present invention would allow the replacement of an autoimmune patient's immune system thereby removing the autoimmune cells of the patient.

Other applications could include xenotransplantation. For instance, the replacement of the human immune system (sensitive to HIV) by a baboon immune system (resistant to HIV). The first AIDS case has been treated with baboon bone marrow stem cells and as to this point the first patients are still alive. The patient did experience some improvement, but at this time baboon hematopoesis has not been observed. These results suggest the need to provide a system in which replacement by the graft cells is facilitated as is set forth in the present invention.

This replacement could be accelerated using baboon Hprt deficient bone marrow cells coupled with 6TG treatment. Bone marrow stem cells from baboon could be cultured, the Hprt gene knocked out or Hprt antisense expressed and the cells reimplanted into a baboon. 6TG treatment will replace the Hprt wildtype cells with the Hprt deficient cells. Hprt deficient baboon cells could then be used in therapy of AIDS patients. Since these baboon stem cells are Hprt deficient, it might be possible to use 6TG selection for bone marrow replacement at earlier stages of the disease.

The present invention also provides a therapy for treating intracellular pathogens (viral, bacteria, parasites) as for example leprosy (Mycobacterium leprae), malaria and AIDS using human cells as set forth in the present invention. In particular, cells of the immune and hematopoietic system which are chronically infected would benefit from the method of the present invention. In one embodiment the therapy utilizes transplantation with Hprt deficient replacement cells that contain the Hprt minigene under control of an intracellular pathogen-dependent promoter. The intracellular pathogen dependent promoter is activated after infection with intracellular pathogen. The uninfected replacement cells would be 6TG resistant. Intracellular pathogen infection will cause expression of Hprt, which results in 6TG sensitivity. Thus, treatment with 6TG will destroy infected cells. This will break the life cycle of the intracellular pathogen.

In a disease such as malaria, in which the red blood cells harbor the pathogen, replacement with a xenodonor stem cell that is resistant to malaria could be used.

As an example, the present invention provides a method for treating HIV infection in a patient by constructing replacement bone marrow stem cells to be resistant to the selecting substance and to contain an HIV-dependent promotor linked to a selecting substance susceptibility gene. The replacement cells can be virus-free cells from the patient which have been genetically engineered to be resistant or the replacement cells.

The constructed stem cells are administered to the patient with AIDS and the patient is treated with the selecting substance to destroy the patient's stem cells allowing the constructed stem cells to repopulate. The patient is then treated periodically thereafter with the selecting substance thereby killing cells derived from the constructed stem cells which have been infected with HIV activating the HIV-dependent promoter linked to the selecting substance susceptibility gene.

The replacement of bone marrow stem cells can also be used in establishing animals, in one embodiment pigs, containing stable xenogeneic (human) bone marrow which can be used for a variety of purposes. For example these animals can accept transplanted human organs. The pigs might be used for in vivo storage of human organs for later transplantation into human recipients. These pigs with human organs can also be used for experimentation where human experimentation is not possible.

These animals will also be a source of human blood derivatives such as antibodies, erythrocytes, platelets, etc. For example, human bone marrow cells from a Lesch-Nyhan patient (Hprt deficiency) will be transplanted into pigs and 6TG selection applied. It is preferable to use newborn pigs that have not yet fully developed their own immune system and to treat them with 6TG before the transfer of human bone marrow cells. Alternatively, donor cells expressing Hprt antisense could be used or donor cells generated by other methods as are known in the art of genetic engineering to replace or inactivate a targeted gene can be used. 6TG will selectively destroy the bone marrow of the pig which will be replaced by the human bone marrow. This will establish human hematopoiesis in pigs. The pig as a host has many advantages such as its physiological similarity to the human, low cost and easy breeding etc. Resulting pig with human hematopoietic tissue will be an important source of human blood derivatives such as immunoglobulines, erythrocytes, platelets. Human immunoglobulines could be produced against specific antigens and used for all possible application e.g. treatment, diagnostics etc. Because such immunoglobulines will be of human origin, there is no concern about their antigenicity. Human erythrocytes and platelets will be used in applications such as transfusion and hematology. These blood particles do not contain DNA and therefore there is low biological risk. Controlled housing of the animals makes it possible to control porcine contaminants and therefore the biohazard will be minimal.

In vivo selection is used to establish an animal (preferentially pig) with human hematopoesis. This animal is useful to produce human blood products such as platelets, lymphocytes, erythrocytes or human immunoglobulines and the like. Antibody (immunoglobulin) production in animals is undertaken as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W. H. Freeman and Co., 1992.

Cell separators are available for clinical use that can be used for isolation of stem and peripheral blood cells and platelets from animals with human hematopoesis (e.g., Nussbaumer et al. 1994). As mentioned above platelets and erythrocytes do not contain DNA and therefore there is minimal biological hazard.

Furthermore, if the aforementioned replacement of the human bone marrow cells with baboon bone marrow stem cells becomes a more widespread AIDS therapy it seem likely that not enough baboons may exist as donors. Pigs are much easier to raise than baboons. One could use the Hprt deficient baboon bone marrow stem cells to replace the pigs hematopoietic system after 6TG treatment as described herein by the method of the present invention. Thus, baboon bone marrow stem cells could be produced in pigs for human AIDS therapy.

Using the aforementioned replacement of the pigs hematopoietic system with a human hematopoietic system will allow these animals to be used for human hematopoietic disease models such as for AIDS research.

As shown in the Examples herein below in vivo selection is used to select for or against specific cell types or tissues or whole embryos or animals. Applicants have shown that positive selection (hyg+/hygromycin and neo+/G418) as well as negative selection (Hprt, 6-thioguanine, 6TG) works in vivo. Analysis of the tissue specific toxicities of the different selection agents show that in general tissues with resistant genotypes are not affected in vivo by the selection compounds. As shown in Example 4 it is possible to select against embryos that were sensitive without compromising the resistant embryos in the pregnant dam.

The present invention allows the selection of distinctive populations of cells in the living organism based on drug metabolism characteristics. This is useful in the treatment of genetic disorders. Particularly, the fact that Hprt deficiency causes resistance of hematopoetic cells to 6TG can be used in the treatment of a variety of hereditary hematological and immune disorders.

Controlled replacement of malfunctional bone marrow cells with healthy bone marrow would cure hereditary hematological diseases particularly anemias (sickle cell anemia, aplastic or hypoplastic anemia, thalassemia), hereditary platelet function disorders, lethal agranulocytopenia, etc. This approach can also be used for treatment of hereditary immune diseases particularly X-linked infantile hypogammaglobulinemia, AIDS, Wiskott-Aldrich syndrome, SCID (ADA deficiency), etc. (Loeb, 1992)

An other embodiment would be to induce cytoablation of virus infected cells with the means of in vivo selection as described herein above in the treatment of AIDS.

The in vivo selection principle can be also used for treatment of congenital and acquired diseases that affect tissues that can be replaced by regeneration such as hematopoietic tissue, liver, and the like. Other candidates of diseases are disorders where repacement of affected tissue will result in cure or decrease of severity, e.g. metabolic diseases, diabetes, phenylketouria, hemophilia, congenital hypercholesterolemia, hepatitis, cystic fibrosis, etc.

Other combination of tissue specific drugs together with appropriate gene therapy allowing negative selection will be useful.

6-thioguanine (6TG) has been used for the treatment of leukemias and as an immunosuppressive agent for several decades. It has been noted that virtually all major current protocols for “average” and “low risk” acute lymphoblastic leukemia (ALL) include daily doses of 6MP as a core component of continuing chemotherapy (Lennard and Lilleyman, 1989). Loss of Hprt has been associated with the resistance of leukemias to 6TG chemotherapy (Brockman, 1974; Davidson and Winter, 1964). However, little or nothing is known about the effect of Hprt status on the tissue-specific toxicity of 6TG in animals. Thus, as shown in the Examples hereinbelow Applicants determined 6TG toxicity in Hprt mutant (Hooper, et al., 1987) and Hprt wildtype mice.

Acute toxicity of 6TG is delayed so that mice treated with the lowest lethal dose survived for 8 to 13 days, and even mice treated with 10 times the approximate lethal dose still survived for 4 days after administration (Table 6). Delayed toxicity of 6TG has been found previously and has been proposed to result from agranulocytosis or thrombocytopenia and to resemble the toxic effects of ionizing radiation (Philips, et al., 1954). It is likely that 6TG has to be metabolized and that the active 6TG metabolites cause damage in proliferating or metabolizing tissue. This may have caused animals death due to liver, hematopoietic and/or gastrointestinal failure. These effects are seen with most of the other purine analog chemotherapeutic agents (Philips, et al., 1954).

Hprt deficiency in the transgenic mice caused marked protection for hematopoietic tissues and intestinal epithelium against 6TG toxicity. Hprt −/− mice given sublethal doses (720 mg/kg or less) of 6TG had normal bone marrow and gastrointestinal tract epithelium. However, after lethal doses (1148 mg/kg or higher) the animals displayed bone marrow lesions similar to treated wildtype mice (Table 8). One possible explanation for this observation is activation of 6TG by an alternate metabolic pathway with lower specificity for 6TG. Although, adenine phosphoribosyltransferase (Aprt) is considered specific for adenine (Blakley, 1986), applicants cannot exclude that high doses of 6TG in these animals might compete with adenine for enzymatic conversion to 6TGMP. Cells deficient in Hprt activity frequently exhibit elevated Aprt activity (Brockman, 1974; Davidson and Winter, 1964).

Evidence suggests that Hprt levels might be important for the efficacy of 6TG cancer treatment. Among 120 children with ALL, patients with lower incorporation of 6TG into 6TG nucleotides show a significant higher risk of relapse than patients with higher incorporation (Lennard and Lilleyman, 1989). Furthermore, among 83 children with untreated ALL, low Hprt activity is correlated with a poorer prognosis (Pieters, et al., 1992). Similarly, among 44 children with ALL, the probability of continuous complete remission was significantly lower in patients with 6TG resistant cells (Pieters, et al., 1991).

Interindividual genetic variability in Hprt activity is an important factor for the efficacy of 6TG therapy and the estimation of expected side effects. 6TG resistance of T cells from Lesch Nyhan syndrome patients was 300 fold higher than cells from normal individuals (Yamanaka, et al., 1985). However, brothers from 3 Lesch-Nyhan patients, that showed Hprt deficiency but no signs of the disease had T cells with 10 fold greater 6TG resistance than normal individuals (Yamanaka, et al., 1985). Thus, Hprt deficiency does not necessarily lead to expression of Lesh-Nyhan syndrome and additional modifying factors are likely to be involved. This finding shows the interindividual variations of 6TG resistance in phenotypically normal people. Furthermore, patients with untreated chronic lymphocytic leukemia had significantly lower Hprt activities than control subjects and the Hprt activities were quite widely dispersed (0-31 nmol/h/mg protein; (Rambotti and Davis, 1981)).

There is also considerable interindividual variability of the frequency of mutations giving rise to 6TG resistance. There is a range of 0.48 to 12 mutations to 6TG resistance per 10⁶ T-lymphocytes from a healthy human population (Davies, et al., 1992). Furthermore, normal, nontransformed cells from patients with cancer prone diseases such as Werners syndrome (Fukuchi, et al., 1990), Ataxia telangiectasia (Cole and Arlett, 1994), Bloom's syndrome (Vijayalaxmi, et al., 1983) and Xeroderma pigmentosum (Cole, et al., 1992) show a significantly elevated frequency of mutations to 6TG resistance. Thus, interindividual differences in 6TG sensitivity of lymphocytes (Lennard and Lilleyman, 1989) and of the frequency of mutations causing 6TG resistance are responsible in part for the variability of the efficacy observed for 6TG chemotherapy.

Applicants found that 6TG treatment in Hprt deficient mice caused bone marrow hyperplasia and extramedullary hematopoesis (Tables 7, 8). It has been found that administration of a single dose of 6TG in Phase I trials to cancer patients resulted in very limited incorporation of 6TG into DNA of bone marrow cells. However, after 5 daily doses of 6TG the guanine residues in the DNA were largely replaced by 6TG is (LePage and Whitecar, 1971), indicating that many cells entered DNA synthesis during the 5-day treatment period. Mitogenic stimulation of lymphocytes leads to a higher purine salvage pathway activity with increased Hprt activities (Peters and Veerkamp, 1979) making Hprt wildtype cells hypersensitive to 6TG killing. However, applicants' study indicated that the hyperplasia caused by 6TG is independent of Hprt activity and occurred at doses 40-fold below those causing toxicity in the Hprt deficient animals. These results suggest that, once 6TG resistant leukemia cells have developed after 6TG chemotherapy, these cells may be driven to proliferate in the presence of 6TG. Thus, administration of 6TG to patients with 6TG resistant cells might actually exacerbate the progression of the disease.

To avoid this, the present invention provides for determining the sensitivity of a patient's lymphocytes to 6TG prior to and during administration of 6TG by lymphocyte cloning or other methods as are known in the art (Dempsey, et al., 1983; Yamanaka, et al., 1985; Veerman and Pieters, 1990). Those patients with 6TG resistant leukemia cells would be identified and their therapy can be altered.

The above discussion provides a factual basis for the use of in vivo selection. The methods used with and the utility of the present invention can be shown by the following non-limiting examples.

### EXAMPLES

General Methods

The present invention provides for improvements to the selection of transgenic animal and cellular (cell lines) models as well as for knockout models. These models are constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson (1991), Capecchi (1989), Davies et al. (1992), Dickinson et al. (1993), Duff and Lincoln (1995), Huxley et al. (1991), Jakobovits et al. (1993), Lamb et al. (1993), Pearson and Choi (1993), Rothstein (1991), Schedl et al. (1993), Strauss et al. (1993) unless stated otherwise. Further, patent applications WO 94/23049, WO 93/14200, WO 94/06908, WO 94/28123 also provide information.

General methods in molecular biology: Standard molecular biology techniques known in the art and not specifically described were generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989). Polymerase chain reaction (PCR) was carried out generally as in PCR Protocols: A Guide To Methods And Applications, Academic Press, San Diego, Calif. (1990). Reactions and manipulations involving nucleic acid techniques, unless stated otherwise, were performed as generally described in Sambrook et al., 1989, Molecular Cloning: A Laboratory Manual, Cold Spring Harbor Laboratory Press, and methodology as set forth in U.S. Pat. Nos. 4,666,828; 4,683,202; 4,801,531; 5,192,659 and 5,272,057 and incorporated herein by reference.

Vectors can be constructed for the present invention by those skilled in the art and should contain all expression elements necessary to achieve the desired transcription of the sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids, liposomes and other recombination vectors. The vectors can also contain elements for use in either procaryotic or eucaryotic host systems. One of ordinary skill in the art will know which host systems are compatible with a particular vector.

The vectors can be introduced into cells or tissues by any one of a variety of known methods within the art. The host cell can be any eucaryotic and procaryotic cells, which can be transformed with the vector and which will support the production of the enzyme. E. coli and Pichia pastoris are host cells in bacterial and yeast (Cregg et al, 1993) preferred embodiments, respectively. Methods for transformation can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or ex vivo. Viral vectors can also be modified with specific receptors or ligands to alter target specificity through receptor mediated events.

### EXAMPLE 1

### Resistance to Hygromycin-B in Vivo by hyg^{R} Expression in Transgenic Mice

Material and Methods

Animals and Drug Treatment

Transgenic mice C57BL/6J-TgN(pPWL512hyg)1Ems carrying hyg^{R} (Johnson et al., 1995) and wild type C57BL/6J were obtained from the Jackson Laboratory, Bar Harbor Me. The animals were kept in SPF conditions, supplied with standard diet and water ad libitum. The room was held at 22° C. with humidity 70% and 12 hours dark-light cycle. Hygromycin-B was purchased from Sigma. The compound was dissolved in sterile water before application and the solution was injected i.p.

Acute Toxicity, Approximate Lethal Dose

The approximate lethal dose of hygromycin B after single i.p. injections was determined according to Deichmann and LeBlanc (1943). This protocol significantly reduces the number of experimental animals, limits unnecessary suffering and complies with current Guidelines of Animal Studies (IARC 1993). Two sets of six month old male mice (22-28 g) were used for all acute toxicity experiments. The animals were treated with a single dose of hygromycin B i.p. at doses that started at 2.7 mg/kg and increased by 50% for each consecutive dose. Control mice were treated with the same volume of sterile saline. Total volume injected was 0.5 ml. The health status and body weights of animals were monitored daily for 10 consecutive days. The lowest dose in the dose response curve at which one mouse died was considered as the approximate lethal dose. Dead or sacrificed moribund animals were necropsied and the organs were stored in 10% buffered neutral formalin until further analysis. The animals were checked twice a day. The maximum theoretical time the animals could be dead before necropsy was performed was 12 hrs. Because animals did not seem moribund when checked it is assumed that actual time was much less.

Histopathological examination and serum biochemical analysis

The cadavers were necropsied and the organs were stored in 10% buffered neutral formalin. The organs were prepared as paraffin-embedded glass slides stained with hematoxylin and eosin and evaluated as per the NTP (National Toxicology Program of NIEHS) standards. A complete cross section of each organ, when possible, was evaluated (liver, spleen, gastrointestinal tract, femoral bone marrow, mandibular and mesenteric lymph nodes, kidney, brain, testes, lungs, and heart). For liver, two cross sections, one of each of the two largest liver lobes were examined. For kidneys, an entire cross section (left longitudinal, right transverse) were evaluated. Lungs had two cross sections (one of each of the two largest lobes). The entire sections on the slides (all fields) were evaluated under blinded conditions for lesions and scored (graded) on a subjective basis compared to control animals. The grades were as follows: 1=minimal, 2=mild, 3=moderate, 4=marked, and 0=no pathological changes. The preparation and evaluation of slides used the NTP criteria and terminology (NIEHS, 1993).

Serum biochemistry parameters analyzed included blood urea nitrogen (SUN), aspartate amino transferase (AST) and alanine amino transferase (ALT). The analyses were performed at Tufts University Veterinary Medical Diagnosis Laboratory with the chemical analyzer Hitachi 747. Blood was collected from the posterior vena cava of hyg^{R} mice that were treated with saline, 803 mg/kg and wildtype mice administered 9 mg/kg hygromycin B i.p.

Northern Blot Analysis

Total RNA samples were isolated from spleen, heart, thigh muscle, lung, kidney, liver, brain and testis by guanidinium thiocyanate-phenol extraction (Chomczynski and Sacchi 1987), separated by electrophoresis in a formaldehyde/agarose gel, and transferred to a nylon HybondN+ membrane (Amersham) by capillary blotting. To compare loading of RNA samples the ethidium bromide stained gels were photographed. The blots were hybridized to a hyg^{R} cDNA probe (Johnson et al. 1995) that was labeled with ³²P dCTP (DuPont) using random oligonucleotide primers (^{T7}QuickPrime, Pharmacia). The autoradiograms were exposed for 48-72 hrs. The bands of hyg^{R} mRNA on autoradiograms and ethidium bromide stained 18S rRNA bands of corresponding samples were analyzed by scanning densitometry with a BioImage (Millipore) system. To compare expression of hyp^{R} mRNA the Relative IOD (integrated optical density) was calculated as total IOD of the autoradiographed band normalized to the intensity of the corresponding 18S ribosomal band visualized by ethidium bromide.

Results

Tissue specific expression of hyg^{R}

The steady state levels of hyg^{R} mRNA in tissues of transgenic mice were examined by northern blot analysis. Total RNA was isolated from spleen, heart, thigh muscle, lung, kidney, liver, brain and testes of male hyg^{R} bearing transgenic and wildtype mice using guanidinium thiocyanate-phenol extraction. The RNA samples were separated by electrophoresis in a formaldehyde/agarose gel, transferred to a nylon membrane by capillary blotting and hybridized with ³²P labeled hyg^{R} cDNA probe. The autoradiograms were evaluated by densitometry. The hyg mRNA reached different levels in different tissues of the hyg^{R} bearing transgenic mice. No signal was detectable in wildtype mice. The highest level of hyg^{R} mRNA expression was detected in the brain. Mid-level expression was detected in skeletal muscle, testis, kidney spleen and liver and somewhat lower levels were detected in heart and lungs.

Acute toxicity, approximate lethal dose

The approximate lethal dose (Deichmann and LeBlanc 1943) of hygromycin B in transgenic hyg^{R} bearing as well as wildtype mice was determined as a measure of the acute toxicity. The mice were treated with single doses of hygromycin B i.p. (2 mice per dose) that increased by 50% for each consecutive dose. The first dose in the increasing sequence of doses at which the mice died was considered the approximate lethal dose. The health status of animals was monitored for ten consecutive days. Hyg^{R} transgenic and wildtype mice tolerated a single i.p. injection of hygromycin B without immediate toxic symptoms or distress. The approximate lethal dose of hygromycin B for wildtype mice was 6 mg/kg (Table 1). Expression of hyg^{R} in transgenic mice caused a substantial increase in resistance to hygromycin B. The approximate lethal dose for hyg^{R} animals was 535 mg/kg, which represents an 89-fold increase over the wildtype strain. Lethal doses in both hyg^{R} expressing transgenic and wild type strains caused the same signs of decreased activity which progressed to lethargy and death. The body weight of animals after lethal doses decreased which was less severe at shorter survival duration after hygromycin treatment (Table 1).

Serum biochemical analysis

Hyg^{R} mice were treated with the lethal dose of 803 mg/kg hygromycin B and wildtype mice were treated with the lethal dose of 9 mg/kg hygromycin B. As a control, hyg^{R} mice were also treated with the nontoxic dose of 9 mg/kg. Serum samples taken 48 hrs after treatment were evaluated for the levels of blood urea nitrogen (BUN), aspartate amino transferase (AST) and alanine amino transferase (ALT). Elevated BUN is associated with dehydration and/or renal insufficiency. Elevated activities of ALT and AST are characteristic for liver damage particularly necrosis, cirrhosis and/or hepatitis. Increased AST levels are also characteristic for muscle trauma, myocardial infarction or myositis.

Administration of 9 mg/kg to wildtype mice resulted in clinically significant elevated levels of BUN, AST, and ALT (Table 2A) suggesting renal injury and hepatocellular damage. In contrast, the same dose given to hyg^{R} mice did not cause any change in those levels (Table 2B). Administration of 803 mg/kg of hygromycin B, a lethal dose to hyg^{R} animals, resulted in elevated levels of BUN, AST and ALT similar to the lethal dose of 9 mg/kg for wildtype mice.

Histopathological examination

Morphological manifestations of hygromycin B tissue specific toxicity in animals treated with lethal doses was assessed by microscopic analysis. The results are summarized in Table 3. Lethal doses of hygromycin B (9 mg/kg) in wildtype animals caused nephrotoxicity typified by tubule eosinophilia, degeneration, and necrosis characterized by cytoplasmic eosinophilia associated with necrosis, loss of tubule cellular and nuclear detail or degeneration, and pyknotic nuclei and fragmentation of cells. These lesions are typical of acute tubular nephrosis. Remaining tissues (liver, spleen, gastrointestinal tract, femoral bone marrow, mandibular and mesenteric lymph nodes, brain, testes, lungs, and heart) were within normal limits. The hyg^{R} mice treated with lethal doses of hygromycin B (803 mg/kg) had liver damage characterized as hepatocellular fatty change, acute inflammation, and hepatocellular necrosis. Liver lesions in the hyg^{R} transgenic mice were typified by hepatocellular necrosis with nuclear pyknosis and loss of cellular detail, acute infiltration with small foci of neutrophils, and fatty change with hepatocellular intracytoplasmic large distinct clear vacuoles that displaced nuclei. These liver lesions are characteristic of acute liver damage. The remaining tissues (spleen, gastrointestinal tract, femoral bone marrow, mandibular and mesenteric lymph nodes, kidney, brain, testes, lungs, and heart) were within normal limits.

### EXAMPLE 2

### Resistance to G418 in Vivo by Expression of neo^{R} in Transgenic Mice

As in Example 1, applicants characterized the tissue specific expression of neoR mRNA and furthermore, investigated acute tissue specific toxicities of G418 in neo^{R}+/+ mice. The neoR mRNA reached highest levels in testis, intermediate in brain, kidney and liver. Low levels were detected in heart skeletal muscle and lungs and almost undetectable low levels in spleen. For acute toxicity determination the animals were treated with a single dose i.p. at concentrations starting at 47 mg/kg and increased thereafter by approximately 50% for each consecutive dose. The neoR expression in transgenic animals caused 10-fold increase of the approximate lethal dose of G418 (Table 4).

The serum biochemical analysis of wild type animals treated with toxic doses of G418 showed significant increase of (BUN) blood urea nitrogen, (CRE) serum creatinine levels and (ALT) alanine amino transferase (Table 5). BUN and CRE levels are diagnostic markers of kidney damage. The increased level of ALT suggests liver injury. In contrast, the animals expressing neo^{R} were protected against toxic effect of G418 and did not show any alteration of serum biochemical parameters (Table 5). In these transgenic animals G418 at the highest dose (1140 mg/kg) caused immediate toxicity resulting in death due to breath failure within 10 minutes after injection. This is probably caused by direct action of G418 on neuromuscular joint as described for neomycin (Chaudhry et al. 1995).

### EXAMPLE 3

### Tissue Specific Toxicities of the Anticancer Drug 6-Thioguanine is Dependent on the Hprt Status in Transgenic Mice

Materials and Methods

Animals and drug treatment

Transgenic Hprt deficient female mice were obtained from Dr. B.Koller (University of North Carolina, N.C.). These mice have a 129/J genetic background and carry a deletion of exons 1 and 2 of the Hprt gene (Hooper, et al., 1987). Control animals, wildtype 129/J mice, were obtained from the Jackson Laboratory (Bar Harbor, Me.). The mice were kept in SPF conditions, provided with standard diet and water ad libitum. Animal care and experimental procedures were carried out in agreement with institutional guidelines.

6-Thioguanine (2-amino-6-merkaptopurine) was purchased from Sigma. The compound was suspended in distilled water and sonicated for 10 minutes before each intraperitoneal (i.p.) injection.

Acute toxicity, approximate lethal dose

The approximate lethal dose of 6TG after single i.p. injections was determined (Deichmann and LeBlanc, 1943). In summary, the animals were treated with a single doses of 6TG i.p. at concentrations starting at 100 mg/kg that increased by 50% for each consecutive dose. Control mice were treated with the same volume of sterile distilled water. Because the LD₅₀ is known for wildtype mice this protocol was modified for doses below 100 mg/kg, and concentrations of only 25 and 50 mg/kg were used to save animals. The lowest dose at which the first animal died was the approximate lethal dose. This dose corresponds to the LD₅₀±30% for most chemicals (Deichmann and LeBlanc, 1943; Deichmann and Mergard, 1948). This protocol significantly reduces the number of experimental animals, limits unnecessary suffering and complies with current Guideline of Animal Studies (IARC recommendation, 1993.). Control mice were treated with the same volume of sterile saline.

Two sets of 8 month old female animals were used for all acute toxicity experiments. The 6TG suspension was injected i.p.. The controls were injected with sterile water. The health status of the animals was observed twice daily and body weight was measured daily for 14 subsequential days. The dead or sacrificed moribund mice were immediately necropsied, and the organs were stored in 10% buffered neutral formalin until further analysis. All survivors were sacrificed with pentobarbital (300 mg/kg, i.p.) 14 days after 6TG administration and necropsies were performed. Necropsies consisted of a gross examination of all external surfaces and orifices and all internal organs.

Histopathological examination and serum biochemical analysis of serum

Tissues were processed as paraffin-embedded tissue glass slides stained with hematoxylin and eosin. These included liver, spleen, gastrointestinal tract, femoral bone marrow, mandibular and mesenteric lymph nodes, kidney, brain, uterus and ovaries, lungs, and heart. Lesions were scored subjectively; —=none, 1=minimal, 2=mild, 3=moderate and 4=marked lesions.

Serum biochemistry parameters analyzed included blood urea nitrogen (BUN), aspartate amino transferase (AST), alanine amino transferase (ALT), creatine kinase (CK), and alkaline phosphatase (AP). The analyses were performed at Tufts University Veterinary Medical Diagnosis Laboratory with the chemical analyser Hitachi 747. Blood was collected from the posterior vena cava of Hprt deficient mice that were either treated with water, 25, 500, or 720 mg/kg 6TG i.p. and wildtype mice administered 25 mg/kg 6TG.

Results

6-thioguanine (6TG) a cytostatic antimetabolite is currently used in clinics to treat cancer, particularly leukemia (PDR, current edition). However, one drawback of its use is the development of 6TG resistance. Hypoxanthine-guanine phosphoribosyl transferase (Hprt) plays a crucial role in the bioactivation of 6TG. Loss of Hprt has been associated with the resistance of leukemia to 6TG chemotherapy. Applicants determined the effect of Hprt status on the tissue specific toxicity of 6TG in vivo in transgenic Hprt deficient mice.

Surprisingly, 6TG induced hypercellular bone marrow occurred in Hprt deficient mice at sublethal doses indicating that 6TG chemotherapy might even exacerbate the progress of 6TG resistant leukemia. Thus, determination of 6TG sensitivity of lymphocytes prior to 6TG chemotherapy and limitation of the treatment to 6TG sensitive patients might improve the efficacy.

Acute toxicity, approximate lethal dose

Hprt deficient and wildtype mice tolerated a single i.p. injection of 6TG without immediate toxic symptoms or distress. The approximate lethal dose of 6TG for wildtype mice was 50 mg/kg (Table 6). Hprt deficiency caused a dramatic increase in resistance to 6TG. The approximate lethal dose for Hprt −/− animals was 1148 mg/kg, which represents a 23-fold increase over the wildtype strain. Lethal doses in both Hprt +/+ and Hprt −/− strains caused the same symptoms including loss of body weight, decreased activity progressing to lethargy, and coma. Sublethal doses in Hprt −/− animals caused no visible effects or changes of body weight. On the other hand, at the 25 mg/kg sublethal dose for the wild type mice a 7% decrease in body weight was found.

Histopathological examination

Some Hprt deficient mice had liver lesions characterized by moderate to marked hepatocellular fatty change typified by large distinct cytoplasmic vacuoles and centrilobular hypertrophy. These lesions did not show any correlation with 6TG dose and did not occur in wildtype control or 6TG treated mice until lethal doses of 225 mg/kg for the fatty change and 150 mg/kg for centrilobular hypertrophy. Therefore, these lesions may be the consequence of Hprt deficiency.

Lesions seen in wildtype and Hprt mutant mice differed strikingly (Tables 7 and 8) for the same doses of 6TG. Starting at 6TG doses of 25 mg/kg Hprt wildtype mice had liver lesions such as necrosis of scattered individual hepatocytes and atrophy of hepatocytes typified by decreased cell size. At higher doses, centrilobular hypertrophy or increased relative size of centrilobular was observed compared to other hepatocytes (Tables 7 and 8). In contrast, Hprt deficient mice did not show necrosis until doses of 507 mg/kg or more, and even at these higher doses necrosis was less severe. In contrast to untreated controls and treated wildtype mice, all Hprt deficient mice treated with sublethal doses of 6TG (100-720 mg/kg) showed slight extramedullary hematopoesis in the liver, which receded at lethal doses.

Wildtype mice even at the lowest dose of 25 mg/kg 6TG experienced depletion and necrosis of hematopoietic tissues, specifically the femoral bone marrow. In addition, cells of the marrow and splenic red pulp were absent or necrotic (Tables 7 and 8). Mandibular and mesenteric lymph nodes had depletions in cell numbers. In contrast, Hprt deficient mice administered doses under 720 mg/kg 6TG had normal to hypercellular bone marrow, spleen, and lymph nodes (Table 7). In fact, all Hprt deficient mice treated with sublethal doses of 6TG from 50 to 720 mg/kg showed hypercellular bone marrow whereas none of the wildtype mice did.

Wildtype mice showed atrophy and/or necrosis of gastrointestinal epithelium (stomach, small and large intestines) such as loss of cells (ulcer/erosion) and decreased thickness of cell layers and height of villi and individual epithelial cells starting at doses of 50 mg/kg 6TG (Tables 7, 8). In contrast, Hprt deficient mice had normal to very minimal changes of gastrointestinal tract epithelium.

Kidneys and other tissues examined in wildtype mice were within normal limits. In contrast, 6TG treated Hprt deficient mice at sublethal doses (150-720 mg/kg) had kidney lesions including scattered dilated renal tubules, focal interstitial inflammation, focal tubule basophilia, and some glomerulopathy characterized by increased numbers of cells (Tables 7, 8).

Hprt deficient mice administered with lethal doses of 6TG (1148 mg/kg or more) had lesions of liver, spleen and bone marrow similar to those observed in the wildtype mice treated with 25 mg/kg or more (Table 8). The kidney lesions in Hprt deficient mice decreased at toxic doses (Table 8).

Serum biochemical analysis

Hprt wildtype mice were treated with the sublethal dose of 25 mg/kg 6TG and Hprt mutant mice were treated with the sublethal doses of 25, 500 and 750 mg/kg 6TG. Serum samples taken 14 days after treatment were evaluated for the levels of blood urea nitrogen (BUN), aspartate amino transferase (AST), alanine amino transferase (ALT), creatine kinase (CK) and alkaline phosphatase (AP). Elevated BUN is associated with dehydratation and/or renal insufficiency. Elevated activities of ALT and AST are characteristic for liver damage particularly necrosis, cirrhosis and/or hepatitis and also muscle trauma or myocardial infarction or myositis. Elevated AP is associated mostly with increased bone marrow metabolism and also with hapatocellular damage during hepatitis. CK is predominantly located in muscles and therefore its increased activities are consequence of muscular trauma, myocardial infarction or myopathic disorders.

Administration of a sublethal dose of 25 mg/kg 6TG resulted in clinically significant elevated levels of AST, and ALT (Table 9A) in Hprt wildtype mice suggesting hepatocellular possibly necrotic damage in those animals. Levels of CK and AP did not increase suggesting that 6TG does not cause muscular damage or bone disorders. There was about a doubling of BUN levels, slightly above the physiological range. Because we did not detect histological evidence for renal toxicity of 6TG in wildtype mice, these slightly elevated levels of BUN could be the result of dehydration caused by impaired gastrointestinal epithelia or due to decreased fluid intake. In contrast, Hprt deficient mice, even after sublethal doses of 720 mg/kg, showed no clinically significant changes of the serum biochemical parameters (Table 9B).

Renal lesions including evidence of tubule and glomerular damage seen only in Hprt −/− mice (Table 8) may be associated with excretion of large amounts of 6TG catabolites such as 6-thiourea in the kidneys. Administration of adenine, purine or 2-chloroadenine causes the “adenine kidney” characterized by precipitation of crystals and induction of lesions (Philips, et al.,1954). However, such crystals were not visible in the present study or after administration of 6MP in mice or man (Philips, et al.,1954). It is possible that the 6TG catabolites do not form visible crystals but still occur and cause irritation and lesions. Renal lesions were mainly developed at sublethal 6TG doses of 150 -720 mg/kg in Hprt deficient mice. Wildtype mice did not survive these doses. Hprt deficient mice at lethal doses (1148 mg/kg or more) also had diminished renal lesions (Table 8) indicating that biodegradation, excretion and/or time might be required for changes seen at sublethal doses. On the other hand, the 6TG catabolic products have not been characterized in Hprt deficient animals. Thus, different catabolic products might be formed in Hprt deficient mice that are more toxic to kidneys.

### EXAMPLE 4

### In Vivo Selection of Embryos

Animals

Transgenic Hprt deficient and wild type C57Bl6/J mice were obtained from the Jackson Laboratory (Bar Harbor, Me.) and bred in applicants, animal facility under SPF conditions. The Hprt−/− mice are of the same genetic background and carry a deletions of exons 1 and 2 of the Hprt gene (Hooper et al. 1987). The animals were housed in standard plastic microisolator cages with wood-chip bedding. The room was maintained at 22° C. and 70% humidity and the light cycle was 12 hours. Water and mouse diet No:5015 (PMI Feeds, Inc., St. Louis, Mo.) was supplied ad libitum. All housing and treatment protocols were reviewed and approved by an institutional Animal Care Committee.

Hprt −/− females (5-8 weeks old) were mated with wildtype males and the presence of vaginal plugs was examined daily. The day when a plug appeared was considered to be day 1 of pregnancy and the male was separated. The pregnant dams were treated with three i.p. injections of 6-thioguanine (20 mg/kg) or in control matings with saline starting at day 4 in 24 hrs intervals. Pregnant mice were examined daily until term. The gender of offsprings was first determined 24 days after delivery and confirmed at the time of weaning (4 weeks of age).

Chemicals

6-Thioguanine (2-amino-6-merkaptopurine) was purchased from Sigma. The compound was suspended in distilled water and sonicated for 10 minutes before each intraperitoneal (i.p.) injection.

Protocol

An experiment was designed to investigate whether one could select against Hprt wildtype embryos with 6-thioguanine. Female mice C57BL6/J Hprt −/− were mated with wildtype Hprt +/o males. Since the Hprt gene is located on the X chromosome the resulting male offspring will be Hprt −/o and therefore resistant to 6-thioguanine (6TG). On the other hand, female offspring will be Hprt +/− and thus sensitive to 6TG. 6TG (Sigma) was injected i.p. in three subsequential doses (20 mg/kg each) in 24 hrs intervals starting on the third day of gestation. Mice were screened two times daily for possible toxic effects.

Results

In agreement with Example 3 herein above, 6TG did not cause any apparent toxic symptoms or discomfort to the dams. The gender of the offsprings was examined during the first 24 hrs post partum. Two 6TG treated dams delivered 6 male offspring. Three control dams handled according the same protocol but without 6TG treatment delivered 4 male and 8 female offspring (Table 10). These results indicate that embryos carrying certain resistance markers can be selected for in vivo.

### EXAMPLE 5

### Bone Marrow Replacement

The major toxic effect of 6-Thioguanine (6TG) is bone marrow suppression. As described herein above, the bone marrow of Hprt deficient animals are resistant to high doses of 6TG. Because of striking differences between 6TG toxicities in the bone marrow of Hprt deficient and wild type animals applicants have used 6TG to select for Hprt deficient bone marrow cells transplanted into wild type animals. The Hprt +/+ bone marrow of the recipient mice is selectively killed by 6TG and only the transplanted Hprt deficient cells are able to survive such treatment and repopulate the bone marrow. As shown below this results in complete replacement of the animals bone marrow cells by the transplanted bone marrow cells.

### Materials and Methods

Animals

All animals for this study were purchased from the Jackson Laboratory (Bar Harbor, Me.). The mice, C57BL6/J Hprt−/− and C57B6/J Hprt+/+ were kept in SPF conditions, provided with standard diet and water ad libitum. Animal care and experimental procedures were carried out in agreement with institutional guidelines.

Bone marrow transplantation

Recipient Hprt+/+ female mice were irradiated with 950 cGy of X-rays 24 hours before transplantation. Irradiation factors: Westinghouse 150 Industrial X-ray Machine producing 130 kvp X-rays, delivered by a self-rectifying tube, inherent filtration 1.65 mm aluminum. Using a current of 8 milliamperes, the intensity obtained at 40 cm distance was 24±2 rads per minute. The mice were exposed in individual sterile polypropylene/polyethylene containers resting on a 24 cm diameter steel turntable. The mice were rotated to ensure a more accurate average value of the irradiated field. The delivered dose was measured for each irradiation with a Victoreen C-r 570 meter.

Bone marrow was isolated from male Hprt−/− mice according to Ramshaw et al. 1995. Briefly, bone marrow was flushed from femora and tibiae with 2 ml of Dulbecco's modified Eagle medium (D-MEM) supplemented with 10% of FBS and 100 units/ml penicillin, 100 mg/ml streptomycin. The isolated cells from the donor mouse were washed with PBS and injected into the tail vein of female Hprt +/+ recipients.

6TG selection

6TG (2-amino-6-mercaptopurine Sigma) was suspended in sterile PBS with a final concentration of 0.5 mg/ml prior to each i.p. application. All mice, recipients of bone marrow transplant and non-recipient controls, were treated with the same 6TG dose regimen. All mice were treated one hour after transplantation of recipients with an initial dose of 6TG at 10 mg/kg body weight. After 7 days the animals were treated with 4 doses of 5 mg/kg b.w. in 48 hrs intervals and followed with 5 days of recovery period.

This regimen was repeated 2 times with doses 5 mg/kg and 3 times with doses of 10 mg/kg with 5 days recovery periods in between treatments. Another control group of transplanted animals was treated with the same amount of sterile PBS.

Results

6TG treated bone marrow (BM) recipients did not show any apparent toxic effect of 6TG (Table 11). Their body weight decreased about 10% during first week after transplantation. The untreated BM recipients exhibited moderate loss of 5% of b.w. and 6TG treatment of non-recipient mice did not result in reduction of body weight during the first week. The subsequential 6TG treatment of bone marrow recipients did not cause any additional loss of body weight. In contrast, the non-recipient controls exhibited typical clinical symptoms of 6TG toxicity such as significant loss of body weight (25%), visible anemia, lethargy progressing to coma and death during 3 weeks of 6TG treatment. The bone marrow recipients not treated with 6TG did not show any clinical symptoms during the experiment.

Transplantation of Hprt deficient bone marrow protects recipients from toxic effects of 6TG indicating that a majority if not all of the hematopoietic system has been replaced by the transplanted bone marrow.

The replacement of Hprt positive bone marrow with Hprt deficient bone marrow was examined by Southern blot analysis. Since recipients animals were females and donor bone marrow was of male origin, we have determined the fraction of cells containing chromosome (Y) in hematopoetic organs (bone marrow, spleen, thymus) and peripheral tissue (tail) in recipient animals. The Y chromosome was detected in 6TG treated and control animals. In comparison to control animals, application of 6TG selection significantly increased engraftment of Hprt deficient bone marrow cells (containing Y chomosome). The presence of Y DNA in peripheral tissues (tail) suggests, that transplanted bone marrow was active and produced circulating white blood cells.

Next Applicants tested whether “in vivo selection” without whole body irradiation can lead to complete bone marrow replacement. The wild type recipient mice were pretreated with 6TG (10 mg/kg twice in 48 hrs interval) or with sterile PBS and the Hprt deficient bone marrow was transplanted. The animals in the experimental group were 6TG pretreated followed with the 6TG in vivo selection protocol (described herein above). The control animals (6TG or PBS pretreated) were treated with sterile PBS buffer instead of 6TG. The degree of bone marrow replacement was evaluated by Southern blot analysis in hematopoetic tissues (bone marrow, spleen, thymus) and blood at the end of the 7th week post transplantation. The results show 100% bone marrow replacement in animals treated with 6TG using the in vivo selection protocol whether or not irradiation was used. In contrast, control groups of transplanted but not 6TG treated animals showed no detectable engraftment measured as presence of Y(donor) DNA in hematopoetic tissues (bone marrow, spleen, thymus) and blood. Irradited but not 6TG treated animals showed 12 to 70% engraftment. Therefore these results demonstrate that in vivo selection allows complete bone marrow replacement in the absence of whole body irradiation and in vivo selection is superior to whole body irradiation for replacement.

TABLE 1
Acute toxicity of hygromycin B in hyg^{R+/+} transgenic mice

| | |
|---|---|
| Wildtype | hyg^{R} |

| dose | survival | body weight^{a} | survival | body weight^{a} |
|---|---|---|---|---|
| [mg/kg] | [days] | [%] | [days] | [%] |
| 0 | surv | 103 ± 6 | surv | 105 ± 5 |
| 2.7 | surv | 107 ± 3 | nd | nd |
| 4 | surv | 104 ± 1 | surv | 102 ± 2 |
| 6 | 3 | 83 ± 2 | surv | 107 ± 4 |
| 9 | 3 | 91 ± 4 | surv | 107 ± 3 |
| 13.5 | 3 | 86 ± 6 | surv | 101 ± 2 |
| 20 | 3 | 83 ± 2 | surv | 105 ± 1 |
| 30 | 3 | 82 ± 2 | surv | 105 ± 3 |
| 46 | nd | nd | surv | 107 ± 5 |
| 69 | nd | nd | surv | 101 ± 3 |
| 104 | nd | nd | surv | 103 ± 2 |
| 155 | nd | nd | surv | 102 ± 2 |
| 233 | nd | nd | surv | 93 ± 5 |
| 358 | nd | nd | surv | 99 ± 3 |
| 535 | nd | nd | 3 | 83 ± 5 |
| 803 | nd | nd | 3.5 | 89 ± 4 |
| 1205 | nd | nd | 2 | 88 ± 2 |
| 1809 | nd | nd | 1 | 97 ± 2 |
| 2709 | nd | nd | 1 | 98 ± 1 |

Mice were treated i.p. with single doses of hygromycin B. controls were treated with the same volume of saline. Body weights were measured daily for 10 days.
^{a}percentage of the body weight on the 10th day before the experiment; surv. animals survived the 10 days period; nd, experiment not done. The values represent averages for two animals ± range.

TABLE 2
Biochemical analysis of serum after treatment of wildtype and hyg^{R}
mice with hygromycin B

| Hygromycin B | | BUN | ALT | AST |
|---|---|---|---|---|
| [mg/kg] | | [mg/dl] | [u/l] | [u/l] |
| Physiol. range | | 10-40 | up to 75 | up to 50 |
| A) | Wildtype | | | |
| | 0 | 20.3 ± 0.5 | 21.0 ± 1.0 | 39.3 ± 1.1 |
| | 9 | 97.7 ± 5.5 | 108 ± 61.5 | 135 ± 29.7 |
| B) | hyg^{R} | | | |
| | 0 | 18.0 ± 1.7 | 22.0 ± 6.2 | 42.0 ± 2.0 |
| | 9 | 24.0 ± 4 | 28.7 ± 15.1 | 43.0 ± 5.2 |
| | 803 | 102 ± 55.1 | 108 ± 66.3 | 102.0 ± 12.7 |

Mice were treated i.p. with single doses of hygromycin-B. controls were treated with the same volume of saline. The serum samples were taken 48 hrs after treatment and frozen until analysis. The values represent averages for 3 animals ± SD. BUN, blood urea nitrogen: ALT, alanine amino transferase: AST, aspartate amino transferase.

TABLE 3
Histopathological analysis of mouse tissues after treatment with
Hygromycin B

| | Wildtype | | hyg^{R} | |
|---|---|---|---|---|
| | Control | Lethal dose | Control | Lethal dose |
| Dose [mg/kg] | 0 | 9 | 0 | 803 |
| Kidney | | | | |
| Tubule eosinophilia | 0 | 1.0 ± 1.2 | 0 | 0 |
| Tubule degeneration | 0 | 2.2 ± 0.4 | 0 | 0 |
| Tubule necrosis | 0 | 1.2 ± 1.1 | 0 | 0 |
| Liver | | | | |
| Necrosis | 0 | 0 | 0 | 2.2 ± 0.4 |
| Acute inflamation | 0 | 0 | 0 | 0.8 ± 1.0 |
| Fatty change | 0 | 0 | 0 | 0.6 ± 0.8 |

Pathological examinations were performed with 5 animals per dose. The tissue samples were taken and preserved in 10% buffered neutral formalin. The values represent the average of lesion severity grade ± standard error (Severity grade 1 = minimal, 2 = mild, 3 = moderate, 4 = marked, 0 = no pathological changes). For grading definition see Materials and Methods section.

TABLE 4
Acute toxicity of G418 in neo +/+ transgenic mice

| | |
|---|---|
| Neo −/− | Neo +/+ |

| dose | survival | body weight^{a} | survival | body weight^{a} |
|---|---|---|---|---|
| [mg/kg] | [days] | [%] | [days] | [%] |
| 0 | surv | 103 ± 6 | surv | 104 ± 4 |
| 47 | surv | 101 ± 1 | surv | nd |
| 68 | surv | 104 ± 4 | surv | 100 ± 1 |
| 103 | 8 | 73 ± 6 | surv | nd |
| 153 | 8 | 73 ± 5 | surv | 102 ± 3 |
| 230 | 4 | 85 ± 2 | surv | 100 ± 2 |
| 345 | 1 | 97 ± 1 | surv | 98 ± 4 |
| 480 | 1 | 99 ± 1 | surv | 100 ± 1 |
| 778 | 1 | 100 ± 2 | surv | 101 ± 1 |
| 1140 | nd | nd | 1 | 100 ± 1 |

Mice were treated i.p. with single doses of G418, controls were treated with the same volume of saline. Body weights were measured daily for 10 days.
^{a}percentage of the body weight on the 10th day of the body weight before the experiment; surv, animals survived the 10 day period; nd, experiment not done. The values represent averages for two animals ± range.

TABLE 5
Biochemical analysis of mouse serum after treatment with G418

| G418 | | BUN | | ALT |
|---|---|---|---|---|
| [mg/kg] | | [mg/dl] | CRE | [u/l] |
| Physiol. range | | 10-40 | [mg/dl] | up to 75 |
| A) | Wildtype | | | |
| | 0 | 20.7 ± 1.7 | 0.23 ± 0.05 | 34.33 ± 8.6 |
| | 335 | 96.0 ± 14.2 | 0.67 ± 0.05 | 102.0 ± 9.4 |
| B) | neo^{R} | | | |
| | 0 | 19.7 ± 2.9 | 0.23 ± 0.05 | 32.7 ± 5.0 |
| | 335 | 22.3 ± 0.9 | 0.23 ± 0.05 | 54.3 ± 14.4 |

Mice were treated i.p. with single doses of G418, controls were treated with the same volume of saline. The serum samples were taken 48 hrs after treatment and frozen until analysis. The values represent averages for 3 animals ± SD. BUN, blood urea nitrogen; CRE, reatinine; AST, aspartate amino transferase.

TABLE 6
Acute toxicity of 6TG in Hprt deficient mice

| | |
|---|---|
| Hprt +/+ | Hprt −/− |

| dose | survival | body weight^{a} | survival | body weight^{a} |
|---|---|---|---|---|
| [mg/kg] | [days] | [%] | [days] | [%] |
| 0 | surv | 105 | surv | 102 |
| 25 | surv | 93 | surv | 101 |
| 50 | 13 | 65 | surv | 102 |
| 100 | 10.5 | 69 | surv | 103 |
| 150 | 8.5 | 76 | surv | 101 |
| 225 | 7 | 80 | surv | 100 |
| 338 | 4 | 88 | surv | 101 |
| 508 | 4 | 85 | surv | 99 |
| 720 | 4 | 80 | surv | 101 |
| 1148 | nd | nd | 8.5 | 69 |
| 1714 | nd | nd | 4 | 93 |
| 2571 | nd | nd | 4 | 95 |

Mice were treated i.p. with single doses of 6TG, controls were treated with the same volume of sterile water. Body weights were measured daily for 14 days.
^{a}percentage of the body weight on the 14th day of the body weight before the experiment; surv, animals survived the 14 days period; ND, experiment not done. The values represent averages for two animals.

TABLE 7
Histopathological analysis of mouse tissues after treatment with different doses of 6TG

| | Doses [mg/kg] | | | | | | | | | | | | | | | | | |
|---|---|---|---|---|---|---|---|---|---|---|---|---|---|---|---|---|---|---|
| Organ | Controls | | 25 | | 50 | | 100 | | 150 | | 225 | | 338 | | 507 | | 720 | |
| Lesion Genotype Hprt | +/+ | −/− | +/+ | −/− | +/+ | −/ − | +/+ | −/− | +/+ | −/− | +/+ | −/− | +/+ | −/− | +/+ | −/− | +/+ | −/− |
| Liver | | | | | | | | | | | | | | | | | | |
| Fatty change | — | 1.5 ± 1.5 | — | — | — | 1.0 ± 1.0 | — | 1 | — | 3 | 1 | 2 | 2 | 2 | 2 | 2.5 ± 0.5 | — | 1.5 ± 0.5 |
| Centrilobular | — | 2.5 ± 0.5 | — | 3 | — | 1.5 ± 0.5 | — | 2 | 2 | 2 | 1 | — | 2 | 1 | 1 | 2.0 ± 0.0 | 1 | 1.0 ± 0.0 |
| hypertrophy | | | | | | | | | | | | | | | | | | |
| Atrophy necrosis | — | — | 2.5 ± 0 5 | — | 3 | — | 2 | — | 2 | — | 2 | — | 1 | — | 1 | 1.0 ± 0.0 | 2 | 0.5 ± 0.5 |
| Extramedullary | — | — | — | — | — | — | — | 1 | — | 1 | — | 1 | — | 1 | — | 1.5 ± 0.5 | — | 1.0 ± 0.0 |
| hematopoesis | | | | | | | | | | | | | | | | | | |
| Bone marrow | | | | | | | | | | | | | | | | | | |
| Depletion | — | — | 3.5 ± 0.5 | — | 4 | — | 4 | — | 4 | — | 3 | — | 4 | — | 3 | — | 4 | — |
| Necrosis | — | — | — | — | 3 | — | 3 | — | 4 | — | 3 | — | 3 | — | 4 | — | 4 | — |
| Hypercellular | — | — | — | — | — | 1.0 ± 1.0 | — | 1 | — | 2 | — | 2 | — | 1 | — | 1.5 ± 0.5 | — | 1.5 ± 0.5 |
| Spleen | | | | | | | | | | | | | | | | | | |
| Lymphoid depletion | — | — | — | — | 1 | — | 1 | — | 2 | — | 3 | — | 2 | 1 | 1 | — | 3 | 0.5 ± 0.5 |
| Necrosis | — | — | — | — | — | — | 2 | — | 2 | — | 4 | — | 1 | — | 2 | — | 3 | — |
| Gastric epithelium | | | | | | | | | | | | | | | | | | |
| Necrosis | — | — | — | — | 1 | — | 3 | — | 1 | — | 1 | — | 3 | — | 1 | — | 1 | — |
| Atrophy | — | — | — | — | 2 | — | 3 | — | 1 | — | 1 | — | 3 | — | 1 | — | 1 | — |
| Intestinal epithelium | | | | | | | | | | | | | | | | | | |
| Necrosis | — | — | — | — | 1 | — | 3 | — | 1 | — | 1 | — | 3 | — | 1 | — | 1 | — |
| Atrophy | — | — | — | — | 2 | — | 3 | — | 1 | — | 1 | — | 3 | — | 1 | — | 1 | — |
| Kidneys | | | | | | | | | | | | | | | | | | |
| Tubule dilatation | — | — | — | — | — | — | — | — | — | 2 | — | 2 | — | 1 | — | 0.5 ± 0.5 | — | 1.5 ± 0.5 |
| Tubule basophilia | — | 1 | — | — | — | — | — | — | — | 1 | — | 2 | — | 1 | — | 1.5 ± 0.5 | — | 1.5 ± 0.5 |
| Intersticial inflamation | — | — | — | 2 | — | — | — | — | — | 2 | — | 2 | — | 2 | — | — | — | 1.5 ± 0.5 |
| Glomerulopathy | — | — | — | 1 | — | — | — | — | — | 3 | — | 2 | — | 1 | — | — | — | 0.5 ± 0.5 |

The tissue samples were taken at necropsy and preserved in 10% buffered neutral formalin. The values represent the severity grade from 1 animal or average of severity grage ± range from 2 animals per dose. (Average severity grade — = no change, 1 = minimal, 2 = mild, 3 = moderate, 4 = marked, pathological changes).

TABLE 8
Summary of histopathological analysis of mouse tissues after treatment with 6TG

| Organ | | | | | | |
|---|---|---|---|---|---|---|
| Lesion | Hprt +/+ | | | Hprt −/− | | |
| | | 6TG sublethal | 6TG lethal | | 6TG sublethal | 6TG lethal |
| Doses | Control | 25 mg/kg | 50-720 mg/kg | Control | 25-720 mg/kg | 1148-2561 mg/kg |
| Number of mice | 2 | 2 | 7 | 2 | 11 | 3 |
| Liver | | | | | | |
| Fatty change | — | — | 0.7 ± 0.9 | 1.5 ± 1.5 | 1.6 ± 0.9 | 3.3 ± 0.6 |
| Centrilobular | — | — | 1.0 ± 0.8 | 2.5 ± 0.5 | 1.2 ± 0.8 | 1.0 ± 1.7 |
| hypertrophy | | | | | | |
| Atrophy necrosis | — | 2.5 ± 0.5 | 1.9 ± 0.7 | — | 0.2 ± 0.4 | 1.3 ± 0.6 |
| Extramedullary | — | — | — | — | 0.8 ± 0.5 | 0.3 ± 0.6 |
| hematopoesis | | | | | | |
| Bone marrow | | | | | | |
| Depletion | — | 3.5 ± 0.5 | 3.7 ± 0.5 | — | — | 2.3 ± 0.6 |
| Necrosis | — | 0.5 ± 0.5 | 3.4 ± 0.5 | — | — | 3.0 ± 0.0 |
| Hypercellular | — | — | — | — | 1.2 ± 0.6 | — |
| Spleen | | | | | | |
| Lymphoid depletion | — | — | 1.9 ± 0.9 | — | 02 ± 0.4 | 3.7 ± 0.6 |
| Necrosis | — | — | 2.0 ± 1.3 | — | — | 2.0 ± 1.7 |
| Gastric epithelium | | | | | | |
| Necrosis | — | — | 1.6 ± 0.9 | — | — | — |
| Atrophy | — | — | 1.7 ± 0.9 | — | — | 0.6 ± 0.6 |
| Intestinal epithelium | | | | | | |
| Necrosis | — | — | 1.6 ± 1.0 | — | — | — |
| Atrophy | — | — | 1.7 ± 0.9 | — | — | 0.6 ± 0.6 |
| Kidneys | | | | | | |
| Tubule dilatation | — | — | — | — | 0.9 ± 0.9 | 0.3 ± 0.6 |
| Tubule basophilia | — | — | — | 0.5 ± 0.5 | 0.9 ± 0.8 | — |
| Intersticial inflamation | — | — | — | — | 1.5 ± 1.0 | — |
| Glomerulopathy | — | — | — | — | 1.0 ± 1.0 | — |

The experiment was performed with 1 or 2 animals per dose (doses see Table 1). The tissue samples were taken at necropsy and preserved in 10% buffered neutral formalin. The values represent the average of lesion severity grade ± standard error. Where two animals in the group the walues are average ± range. (Average severity grade 1 = minimal, 2 = mild, 3 = moderate, 4 = marked, — no pathological changes).

TABLE 9
Biochemical analysis of mouse serum after treatment with 6TG

| 6TG | BUN | AST. | ALT | CK | AP |
|---|---|---|---|---|---|
| [mg/kg] | [mg/dl] | [u/l] | [u/l] | [u/l] | [u/l] |
| Physiol. range | 10-40 | up to 75 | up to 50 | up to 50 | up to 100 |
| A) Hprt +/+ | | | | | |
| 0 | 21.7 ± 5.0 | 49.7 ± 9.5 | 21.0 ± 6.1 | 68.3 ± 17.4 | 81.3 ± 15.3 |
| 25 | 45.0 ± 24.6 | 256.3 ± 73.9 | 197 ± 66.8 | 73.3 ± 13.9 | 50.0 ± 33.4 |
| B) Hprt −/− | | | | | |
| 0 | 18.7 ± 1.5 | 46.0 ± 6.1 | 29.7 ± 11.9 | 55.7 ± 26.0 | 68.3 ± 33.4 |
| 25 | 18.5 ± 0.6 | 53.3 ± 17.5 | 28.7 ± 14.9 | 67.7 ± 41.2 | 78.7 ± 22.5 |
| 500 | 18.0 ± 1.0 | 50. ± 11.8 | 14.3 ± 3.5 | 52.0 ± 23.3 | 71.0 ± 38.0 |
| 720 | 20.0 ± 2.7 | 43.3 ± 14.0 | 13.7 ± 1.5 | 37.3 ± 11.6 | 78.7 ± 20.5 |

Mice were treated i.p. with single doses of 6TG, control animals were treated with the same volume of sterile distilled wather. The serum samples were taken 14 days after treatment and frozen until analysis. The values represent the averages ± SD from 3 animals. BUN, blood urea nitrogen; AST, aspartate amino transferase; ALT, alanine amino transferase; CK, creatinine kinase; AP, alkaline phosphatase.

TABLE 10
In vivo selection of Hprt deficient embryos

| | | |
|---|---|---|
| Treatment: | Control | 6-thioguanine |

| | | | | |
|---|---|---|---|---|
| Offspring | ♂ | ♀ | ♂ | ♀ |

| | | | | | |
|---|---|---|---|---|---|
| Dam | | | Dam | | |
| 1C | 1 | 4 | 1T | 4 | 0 |
| 2C | 1 | 1 | 2T | 2 | 0 |
| 3C | 2 | 3 | | | |

TABLE 11
Body weight and survival of bone marrow recipients

Bone marrow recipients

| Week | no 6TG | +6TG | Controls +6TG |
|---|---|---|---|
| 1 | 95 | 91 | 99 |
| 2 | 95 | 89 | 100 |
| 3 | 99 | 90 | 86* |
| 4 | 101 | 90 | 79* |
| 5 | 107 | 88 | — |
| 6 | 115 | 93 | — |

*These mice died.

TABLE 12
Available selectable markers with application for in vivo selection

| Gene | Selective drug | |
|---|---|---|
| Negative selectable markers | | Mechanism of action |
| Hprt (hypoxanthine phosphorybosyl transferase) | 6-Tioguanine (6TG) | Bioactivation of 6TG |
| HSV-TK (thimidine kinase) | Ganciclovir | Bioactivation of ganciclovir |
| Positive selectable markers | | Mechanism of action (Reference) |
| CAD (Carbamoyl-phosphate | N-(phosphonacetyl)-L-aspartate (PALA) | Nonfunctional analog of aspartate |
| synthetase-aspartate | | transcarbamylase (Wahl et al. 1979, J. Biol |
| transcarbamoylase-dihydroorotase) | | Chem. 254: 8679-89) |
| DHFR (dihydropholate reductase) | Methotrexate (MTX) | Reduced activity to normaly toxic MTX |
| neo^{R} (neomycine phosphotransferase) | G418 | Protects from G418 interference with 80S |
| | | ribosomal subunit |
| hyg^{R} (hygromycin-B phosphotransferase) | Hygromycin-B | Protects from hygromycin-B interference |
| | | with ribosomal translocation |
| pac (puromycin N-acetyl transferase) | Puromycin | Protects from puromycin interference with |
| | | 80S ribosomal subunit |
| ble (phleomycin binding protein) | Phleomycin | Binds phleomycin to protect from DNA |
| | | strand breaking action |
| zeo (zeocin binding protein) | Zeocin | Binds zeocin to protect from DNA strand |
| | | breaking action |
| mdrl (multiple drug resistence protein) | Vinca alcaloids | Energy dependent efflux pump eliminating |
| | | inracellular toxins |
| hisD (histidinol dehydrogenase) | Histidinol | Protect from histidinol inhibition of |
| histidyl- | | tRNA synthase |

Modified and actualized from Eglitis M. A. Positive selectable markers for use with mammalian cells in cuture. Human Gene Therapy 2: 195-201 (1991)

Throughout this application, various publications, including United States patents, are referenced by author and year for publications and by number for patents. Full citations for the reference publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

Aubrecht et al., Tissue specific toxicities of the anticancer drug 6-thioguanine is dependent on the Hprt status in transgenic mice. Submitted,

Abou el-Ezz et al., A minimal conditioning approach to achieve stable multilineage mouse plus rat chimerism. Transplant Immunology, 3(2):98-106, 1995.

Bleyer, Cancer chemotherapy in infants and children, Pediatric Clinics of North America. 32: 557-74, 1985.

Blakley, Purine phosphoribosyl transferases. In: G. L. Zubay (ed.) Biochemistry, pp. 710-721. London: The Benjamin/Cummings Publ., 1986.

Brockman, R. W. Resistance to purine analogs, Biochemical Pharmacology. Supplement: 107-117, 1974.

Burke and Olson, “Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270 (1991).

Calabresi and Parks Jr., Chemotherapy of neoplastic diseases. In: A. G. Gillman, L. S. Goodman, T. W. Rall, and F. Murao (eds.), Goodman and Gillman's the pharmacological basis of therapeutics, pp. 1240-1308. New York: Macmillian Publ., 1985

Capecchi, Altering the genome by homologous recombination Science 244:1288-1292, 1989.

Caskey and Kruh, The HPRT locus, Cell. 16: 1-9, 1979.

Chaudhry, et al., Neurochemical evidence that [Ca2+]o antagonizes the effect of neomycin on acetylcholine release from mouse hemidiaphragm preparation: an attempt to assess the margin to safety. Acta Anaesthesiologica Scandinavica 39: 494-7, 1995.

Chomczynski and Sacchi, Single step method of RNA isolation by acid quaninium thiocyanate-phenol chlorophorm extreaction. Anal. Biochem, 1987. 162:156-59

Cole and Arlett, Cloning efficiency and spontaneous mutant frequency in circulating T-lymphocytes in ataxia-telangiectasia patients, International Journal of Radiation Biology. 66: S123-31, 1994.

Cole, et al., Elevated hprt mutant frequency in circulating T-lymphocytes of xeroderma pigmentosum patients, Mutation Research. 273: 171-8, 1992.

Cozzi and White, The generation of transgenic pigs as potential organ donors for humans. [Review]. Nature Medicine, 1995. 1(9):964-6.

Cregg J M, Vedvick T S, Raschke W C: Recent Advances in the Expression of Foreign Genes in Pichia pastoris, Bio/Technology 11:905-910, 1993

Davidson and Winter, Purine nucleotide pyrophosphorylases in 6-mercaptopurine-sensitive and -resistant human leukemias, Cancer Research. 24: 261-267, 1964.

Davies, et al., Thioguanine-resistant mutant frequency in T-lymphocytes from a healthy human population, Mutation Research. 265: 165-71, 1992.

Davies et al., Targeted alterations in yeast artificial chromosomes for inter-species gene transfer, Nucleic Acids Research, 20(11): 2693-2698, 1992.

Deichmann and LeBlanc, Determination of the approximate lethal dose with about six animals, Journal of Industrial Hygiene and Toxicology. 25: 415-417, 1943.

Deichmann and Mergard, Comparative evaluation of methods employed to express the degree of toxicity of a compound, Journal of Industrial Hygiene and Toxicology. 30: 373-378, 1948.

Dempsey, et al., Detection of the carrier state for an X-linked disorder, the Lesch-Nyhan syndrome, by the use of lymphocyte cloning, Human Genetics. 64: 288-90, 1983.

Dickinson et al., High frequency gene targeting using insertional vectors, Human Molecular Genetics, 2(8): 1299-1302, 1993.

Duff and Lincoln, Insertion of a pathogenic mutation into a yeast artificial chromosome containing the human APP gene and expression in ES cells. In: Research Advances in Alzheimer's Disease and Related Disorders, 1995.

Elion, Biochemistry and pharmacology of purine analogues, Federation Proceedings. 26: 898-904, 1967.

Epstein and Preisler, Protection of normal hematopoietic stem cells from the toxicity of purine base analogs: in vivo application, Cancer Treatment Reports. 68: 1153-6, 1984.

Fodor et al., Expression of a functional human complement inhibitor in a transgenic pig as a model for the prevention of xenogeneic hyperacute organ rejection. Proceedings of the National Academy of Sciences of the United States of America, 91(23):11153-7, 1994.

Fukuchi, et al., Increased frequency of 6-thioguanine-resistant peripheral blood lymphocytes in Werner syndrome patients, Human Genetics. 84: 249-52, 1990.

Gambiez et al., The role of natural IgM in the hyperacute rejection of discordant heart xenografts. Transplantation, 54(4):577-83, 1992.

Gilboa, et al., Transfer and expression of cloned genes using retroviral vectors. BioTechniques 4(6):504-512, 1986.

Goodman and Gilman's The Pharmacological basis of therapeutics. 7th Edition. MacMillan Publishing Company, New York.

Harlow and Lane, “Antibody a laboratory manual” Cold Spring Harbor Laboratory, New York 1988

Hashimoto, et al., Biochemical basis of the prevention of 6-thiopurine toxicity by the nucleobases, hypoxanthine and adenine, Leukemia Research. 14: 1061-6, 1990.

Huxley et al., The human HPRT gene on a yeast artificial chromosome is functional when transferred to mouse cells by cell fusion, Genomics, 9:742-750, 1991.

Hooper, et al., HPRT-deficient (Lesch-Nyhan) mouse embryos derived from germline colonization by cultured cells, Nature. 326: 292-5, 1987.

IARC recommendation on LD₅₀ testing, ILAR News. 35: 56-58, 1993.

Ildstad et al., Cross-species bone marrow transplantation: evidence for-tolerance induction, stem cell engraftment, and maturation of T lymphocytes in a xenogeneic stromal environment (rat----mouse). Journal of Experimental Medicine, 174(2):467-78, 1991.

Jakobovits et al., Germ-line transmission and expression of a human-derived yeast artificial chromosome, Nature, 362: 255-261, 1993.

Johnson et al., Plasma exchange, splenectomy, and cyclophosphamide for natural antibody removal: kinetics and xenograft survival. Transplantation Proceedings, 24(2):701, 1992.

Johnson et al. Transgenic mouse for the preparation of hygromycin-resistant primary embryonic fibroblast feeder layers for embryonic stem cells. Nucleic Acid Research, 23(7):1273-5, 1995.

Kaufman, et al., Xenotransplantation. [Review]. Annual Review of Immunology, 13:339-67, 1995.

Lamb and Gearhart, YAC transgenics and the study of genetics and human disease. Current Opinion in Genetics and Development, 5:342-8, 1995.

Lamb et al., Introduction and expression of the 400 kilobase precursor amyloid protein gene in transgenic mice, Nature Genetics, 5: 22-29, 1993.

LePage and Whitecar, Pharmacology of 6-thioguanine in man, Cancer Research. 31: 1627-31, 1971.

LePage, Basis biochemical effects and mechanism of action of 6-thioguanine, Cancer Research. 23: 1202-1206, 1963.

Lennard and Lilleyman, Variable mercaptopurine metabolism and treatment outcome in childhood lymphoblastic leukemia [published erratum appears in J Clin Oncol 1990 Mar;8(3):567], Journal of Clinical Oncology. 7: 1816-23, 1989.

Ling, et al., Consequences of 6-thioguanine incorporation into DNA on polymerase, ligase, and endonuclease reactions, Molecular Pharmacology. 42: 802-7, 1992.

Loeb et al. Professional guide to diseases. Fourth edition. 1992, Springhouse Corp., Springhouse, Pa.

Loo and Nelson, Purine antimetabolites. In: J. F. Holland and E. Frei III (eds.), Cancer Medicine, pp. 790-801. New York: Lea & Febiger, 1982

Nussbaumer et al., “PLT-30: an improvement for platelet collection on the Baxter CS-3000 Plus” Journal of Clinical Apheresis 9(4): 235-9, 1994.

Pan and Nelson, Characterization of the DNA damage in 6-thioguanine-treated cells, Biochemical Pharmacology. 40: 1063-9, 1990.

Pearson and Choi, Expression of the human β-amyloid precursor protein gene from a heast artificial chromosome in transgenic mice. Proc. Natl. Scad. Sci. USA, 90:10578-82, 1993.

Peters and Veerkamp, Concentration, synthesis and utilization of phosphoribosylpyrophosphate in lymphocytes of five mammalian species, International Journal of Biochemistry. 10: 885-8, 1979.

Philips, et al., The toxic effect of 6-mercaptopurine and related compounds, Annals of New York Academy of Science. 60: 283-296, 1954.

Pierson et al., Xenogeneic skin graft rejection is especially dependent on CD4+ T cells. Journal of Experimental Medicine, 170(3):991-6, 1989.

Pieters, et al., Hypoxanthine-guanine phosphoribosyl-transferase in childhood leukemia: relation with immunophenotype, in vitro drug resistance and clinical prognosis, International Journal of Cancer. 51: 213-7, 1992.

Pieters, et al., Relation of cellular drug resistance to long-term clinical outcome in childhood acute lymphoblastic leukaemia, Lancet. 338: 399-403, 1991.

Platt et al., Endothelial cell antigens recognized by xenoreactive human natural antibodies. Transplantation, 1990. 50(5):817-22.

Rambotti and Davis, Hypoxanthine-guanine phosphoribosyltransferase activity in normal and leukaemic lymphocytes, British Journal of Haematology. 49: 23-8, 1981.

Ricordi et al., Xenotransplantation of hematopoetic cells resistant to HIV as a potential treatment for patients with AIDS, Transplantation proceedings 26(3) 1302-3, 1996

Rothstein, Targeting, disruption, replacement, and allele rescue: integrative DNA transformation in yeast, In Methods in Enzymology, Vol. 194, “Guide to Yeast Genetics and Molecular Biology”, eds. C. Guthrie and G. Fink, Academic Press, Inc., Chap. 19, pp. 281-301 (1991).

Sato, et al., Chemical mutagenesis at the phosphoribosyltransferase locus in cultured human lymphoblasts, Proceedings of the National Academy of Sciences of the United States of America. 69: 1244-8, 1972.

Schedl et al., A yeast artificial chromosome covering the tyrosinase gene confers copy number-dependent expression in transgenic mice, Nature, 362:258-261, 1993.

Sharabi et al., Specific tolerance induction across a xenogeneic barrier: production of mixed rat/mouse lymphohematopoietic chimeras using a nonlethal preparative regimen. Journal of Experimental Medicine, 1990. 172(l) :195-202.

Siminovitch, On the nature of hereditable variation in cultured somatic cells, Cell. 7: 1-11, 1976.

Strauss et al., Germ line transmission of a yeast artificial chromosome spanning the murine α₁ (I) collagen locus, Science, 259: 1904-1907,1993.

Tanaka et al., Xenotransplantation from pig to cynomolgus monkey: the potential for overcoming xenograft rejection through induction of chimerism. Transplantation Proceedings, 1994. 26(3):1326-7.

Veerman and Pieters, Drug sensitivity assays in leukaemia and lymphoma, British Journal of Haematology. 74: 381-384, 1990.

Vijayalaxmi, et al., Bloom's syndrome: evidence for an increased mutation frequency in vivo, Science. 221: 851-3, 1983.

Weisman et al., Soluble human complement receptor type 1: in vivo inhibitor of complement suppressing post-ischemic myocardial inflammation and necrosis. Science, 1990. 249(4965):146-51.

Yamanaka, et al., Evaluation of the severity of hypoxanthine-guanine phosphoribosyltransferase deficiency using viable T cells, Human Heredity. 35: 358-63, 1985.

Zanjani et al., Successful stable xenograft of human fetal hemopoietic cells in preimmune fetal sheep. Transactions of the Association of American Physicians, 104:181-6, 1991.

Zanjani et al., Retention of long-term repopulating ability of xenogeneic transplanted purified adult human bone marrow hematopoietic stem cells in sheep. Journal of Laboratory & Clinical Medicine, 126(1):24-8, 1995.

What is claimed is:

1. A method of selecting for cells in vivo by
providing in vivo cells which are resistant to a selecting substance and cells which are non-resistant to the selecting substance; and
selecting in vivo for the resistant cells by providing the selecting substance in vivo at a dose nontoxic to the resistant cells and toxic to the non-resistant cells thereby replacing non-resistant cells by resistant replacement cells.

2. A method of selecting for cells in vivo as set forth in claim 1 wherein the resistant cells are genetically engineered to be resistant.

3. A method of selecting for cells in vivo as set forth in claim 1 wherein the non-resistant cells are genetically engineered to be non-resistant.

4. A method of selecting for cells in vivo as set forth in claim 1 wherein the non-resistant cells are identified in a cell population in a host that carries at least one gene making the host cell population selectively susceptible to the selecting substance.

5. A method of selecting for cells in vivo as set forth in claim 1 wherein the resistant cells are genetically resistant and are administered to a host that carries at least one gene making the host cell population selectively susceptible to the selecting substance.

6. The method of claim 1 wherein the resistant replacement cells have a gene selected from the group set forth in Table 1.

7. The method of claim 6 wherein the resistant cells are Hprt−.

8. The method of claim 7 wherein the resistant cells are cells from a Lesch-Nyhan patient.

9. The method of claim 2 wherein the resistant cells have been genetically engineered to be resistant by inserting a gene selected from the group consisting of Hprt−, neo+ and hyg+.

10. The method of claim 2 wherein the cells being genetically engineered are from the host.

11. The method of claim 1 wherein the resistant replacement cells are xenotransplants.

12. The method of claim 11 wherein the cells are baboon cells.

13. The method of claim 11 wherein the cells are porcine cells.

14. The method of claim 2 wherein the cells being genetically engineered have been genetically engineered to also carry at least one other transgene.

15. The method of claim 3 wherein the cells being genetically engineered have been genetically engineered to also carry at least one other transgene.

16. The method of claim 1 wherein the selecting substance is selected from the group consisting of 6-thioguanine (6TG), 8-azaguanine, 6-mercaptopurine and other purine analogs, hygromycin-B, G418 and other neomycin analogs, diphtheria toxin, ouabain, anti-HLA antibody and other agents set forth in Table 1.

17. The method of claim 16 wherein the selecting substance is 6-thioguanine (6TG).

18. The method of claim 3 wherein the gene conferring susceptibility is selected from the group consisting of Hprt+, neo+ and hyg+.

19. The method of claim 18 wherein the gene conferring susceptibility is Hprt+.

20. The method of claim 1 wherein said selecting step is performed in utero.

21. The method of claim 1 wherein said selecting step is performed for embryos in vitro.

22. The method of claim 1 wherein the cells are bone marrow cells.

23. The method of claim 1 wherein said selecting in vivo for the resistant cells by providing the selecting substance in vivo step is repeated.

24. A method according to claim 20 further characterized to select for transgenic animals in utero by
providing fertilized eggs that have a non-resistant genotype;
injecting the fertilized eggs with a vector carrying a transgene and means for providing resistance;
implanting the injected fertilized eggs in a female with a resistant genotype; and
treating the pregnant female with the selecting substance thereby selecting for embryos that are resistant and which carry the transgene and selecting against embryos that are non-resistant and which do not carry the transgene.

25. The method of claim 24 wherein the means for providing resistance includes the antisense sequence for the gene controlling susceptibility to the selecting substance in the vector.

26. The method of claim 24 wherein the selecting substance is 6TG, the resistant genotype is Hprt− and the non-resistant genotype is Hprt+.

27. The method of claim 24 wherein the selecting substance is G418, the resistant genotype is neo+ and the non-resistant genotype is wild type.

28. The method of claim 24 wherein the selecting substance is hygromycin-B, the resistant genotype is hyg+ and the non-resistant genotype is wild type.

29. A method according to claim 20 further characterized to select for germ-line transmission events in chimeras by
establishing chimeric animals which carry a transgene linked to a gene conferring resistance to a selecting substance;
mating the chimeric animals to test for germ-line transmission; and
treating the chimeric animal with possible germ-line transmission of the transgene linked to the selecting substance targeted to germ cells thereby selecting for cells that carry the gene conferring resistance to the selecting substance and which carry the transgene and selecting against cells that do not carry the transgene.

30. The method of claim 29 wherein the selecting substance is 6TG and the resistant gene is Hprt+.

31. The method of claim 29 wherein the selecting substance is G418 and the resistant gene is Neo^{R}.

32. The method of claim 29 wherein the selecting substance is hygromycin-B and the resistant gene is Hyg^{R}.

33. A method according to claim 20 further characterized to establish genetically engineered xenotransplant donors by selecting for transgenic animals having a genotype allowing graft acceptance by a human host including the steps of
establishing pregnant females with the possibility of carrying at least one fertilized egg with a genotype allowing graft acceptance by a human host linked to a gene conferring resistance to a selecting substance; and
treating the female carrying the fertilized eggs with the selecting substance thereby selecting for embryos that carry the gene conferring resistance to a selecting substance and which therefore carry the genotype allowing graft acceptance by a human host and selecting against embryos that do not carry the transgene.

34. A method according to claim 22 further characterized to establish selecting for bone marrow transplant cells in vivo by
identifying a gene in a bone marrow recipient that makes the recipient's bone marrow population selectively susceptible to a selecting substance;
administering to the recipient replacement bone marrow that is not susceptible to the selecting substance; and
administering to the host the selecting substance at a dosage that is selectively toxic for the recipient's bone marrow but not the replacement bone marrow.

35. The method as set forth in claim 34 wherein the replacement bone marrow cells are marrow stem cells from a Lesch-Nyhan patient.

36. The method as set forth in claim 35 wherein the replacement bone marrow is marrow stem cells from a Lesch-Nyhan patient that have been transformed with a MDR1 gene conferring multiple drug resistance to chemotherapy drugs.

37. The method as set forth in claim 34 wherein the recipient is a pig.

38. A method according to claim 34 wherein the recipient has an autoimmune syndrome.

39. A method according to claim 21 further characterized to establish selecting for bone marrow transplant cells in vivo for treatment of patients infected with an intracellular pathogen by
constructing bone marrow stem cells to be resistant to the selectable substance and to contain an intracellular pathogen-dependent promotor linked to a gene making cells non-resistent;
administered the constructed stem cells to a patient who is intracellular pathogen-positive;
treating the patients with the selectable substance to destroy the patients stem cells whereby the constructed stem cells will repopulate; and
treating periodically thereafter with the selectable substance thereby killing cells derived from the constructed stem cells which have been infected with the intracellular pathogen activating the intracellular pathogen-dependent promotor linked to the selectable substance susceptibility gene.

40. The method of claim 39 wherein the intracellular pathogen is HIV.

41. The method of claim 39 wherein the selectable substance is 6TG, the resistant gene is Hprt− and the susceptibility gene is Hprt+.

42. The method of claim 39 wherein the selectable substance is G418, the resistant gene is neo+ and the susceptibility gene is wild type.

43. The method of claim 39 wherein the selectable substance is hygromycin-B, the resistant gene is hyg+ and the susceptibility gene is wild type.

44. The method of claim 39 wherein the replacement cells are from the host and have been genetically engineered to be resistant to the selectable substance.

45. The method of claim 39 wherein the replacement cells are xenotransplants which are HIV resistant.

46. The method of claim 44 wherein the replacement cells have been genetically engineered to also carry a second transgene.

47. A transgenic animal selected in utero by providing in utero embryos resistant to a selecting substance and embryos non-resistant to the selecting substance; and
selecting in utero for the resistant embryos by providing the selecting substance in utero at a dose nontoxic to the resistant embryos and toxic to the non-resistant embryos.

48. A method of determining patients who should not receieve 6-thioguanine (6TG) and other purine analogs as chemotherapy by
testing isolated lymphocytes for Hprt enzyme activity.

49. The method of claim 1 for establishing an animal having a human hematopoetic system by
replacing the animal hematopoetic system by a human hematopeoetic system.

50. An animal having a human hematopoetic system made by the process of claim 49.
